(19) United States
(12) Patent Application Publication
(10) Pub. No.: US 2025/0361528 A1
(43) Pub. Date: Nov. 27, 2025

(54) NOVEL ZINC FINGER FUSION PROTEINS FOR NUCLEOBASE EDITING

(71) Applicant: Sangamo Therapeutics, Inc., Brisbane, CA (US)

(72) Inventors: Friedrich A. Fauser, Fremont, CA (US); Jeffrey C. Miller, San Rafael, CA (US); Sebastian Arangundy, Albany, CA (US)

(73) Assignee: Sangamo Therapeutics, Inc., Brisbane, CA (US)

(21) Appl. No.: 18/722,513

(22) PCT Filed: Dec. 22, 2022
(86) PCT No.: PCT/US2022/082232

§ 371 Date: Jun. 20, 2024

Related U.S. Application Data
(60) Provisional application No. 63/292,817, filed on Dec. 22, 2021.

(51) Int. Cl. C12N 15/90; C07K 14/47; C12N 9/22; C12N 9/78

(52) U.S. Cl. CPC C12N 15/90; C07K 14/47; C12N 9/22; C12N 9/78; C12Y 301/21004; C12Y 305/04005; C07K 2319/00

(57) ABSTRACT

Provided herein are base editor systems comprising fusion proteins that comprise zinc finger protein and cytidine deaminase domains, as well as methods of using the base editor systems. The systems can be used to specifically alter a single base pair in a target DNA sequence.

### CROSS REFERENCE TO RELATED APPLICATIONS

The present application is a U.S. national phase application under 35 U.S.C. § 371 of PCT/US22/82232 filed on Dec. 22, 2022 and entitled “NOVEL ZINC FINGER FUSION PROTEINS FOR NUCLEOBASE EDITING,” which claims priority from U.S. Provisional Application 63/292,817, filed Dec. 22, 2021, the entire contents of each of which applications are incorporated by reference herein.

### SEQUENCE LISTING

The instant application contains a Sequence Listing that has been submitted electronically in XML format. The Sequence Listing is hereby incorporated by reference in its entirety. The XML copy, created on Aug. 20, 2024, is named 91355_09100.xml and is 164 kilobytes in size.

### BACKGROUND OF THE INVENTION

Precision DNA editing of single bases has various applications in treating and understanding disorders such as genetic diseases. For example, knock-out of one or more genes can be achieved by converting regular codons into stop codons, or by mutating splice acceptor sites to introduce exon skipping and/or frameshift mutations. Further, DNA point mutations are associated with a wide range of disorders. Single base editing can be used to correct deleterious mutations or to introduce beneficial genetic modifications.

Cytidine deaminases convert the nucleobase cytosine to thymine (or the nucleoside deoxycytidine to thymidine). These enzymes function in the pyrimidine salvage pathway, predominantly operating on single-stranded DNA to convert cytosine into uracil, which is subsequently replaced by a thymine base during DNA replication or repair. A cytidine deaminase identified in the bacterium Burkholderia cenocepacia, DddA, can catalyze the deamination of cytosine to uracil within double-stranded DNA. DddA thus bypasses the requirement for unwinding of the dsDNA to ssDNA (Mok et al., Nature (2020) 583:631-7). While the Mok study reports C to T base editing at the human CCR5 locus with a DddA-derived cytosine base editor fused to transcription activator-like effector (TALE) proteins, it is unclear how broadly this approach is applicable. Further, new deaminases that operate on double-stranded DNA may have improved or altered base editing activity compared to DddA.

Thus, there continues to be a need to develop precise base editing systems for the prevention and treatment of numerous diseases.

### SUMMARY OF THE INVENTION

The present disclosure provides zinc finger protein (ZFP) based nucleobase editing systems and uses thereof. In one aspect, the present disclosure provides a system for changing a cytosine to a thymine in the genome of a cell (e.g., a eukaryotic or prokaryotic cell, wherein the eukaryotic cell may be a mammalian cell such as a human cell, or a plant cell), comprising a first fusion protein and a second fusion protein, or first and second expression constructs for expressing the first and second fusion proteins, respectively, wherein a) the first fusion protein comprises: i) a first zinc finger protein (ZFP) domain that binds to a first sequence in a target genomic region in the cell, and ii) a first portion of a cytidine deaminase polypeptide (e.g., wherein the cytidine deaminase is a toxin-derived deaminase (TDD) comprising an amino acid sequence at least 90%, 92%, 95%, 96%, 97%, 98%, or 99% identical to any one of SEQ ID NOs: 13-24); b) the second fusion protein comprises: i) a second ZFP domain that binds to a second sequence in the target genomic region, and ii) a second portion of the cytidine deaminase polypeptide; and c) binding of the first fusion protein and the second fusion protein to the target genomic region results in dimerization of the first and second portions, wherein the dimerized portions form an active cytidine deaminase capable of changing a cytosine to a uracil in the target genomic region. In some embodiments, the first and second portions lack cytidine deaminase activity on their own. In some embodiments, the first and second portions form an active cytidine deaminase that comprises an amino acid sequence at least 90% identical to any one of SEQ ID NOs: 13-24. In some embodiments, the first and second portions form an active cytidine deaminase that comprises the amino acid sequence of any one of SEQ ID NOs: 13-24. In some embodiments, the target genomic region may be specific to a particular allele of a gene in the cell. In some embodiments, the targeted cytosine may be between the proximal ends of the first sequence and the second sequence in the target genomic region, optionally wherein the proximal ends are no more than 100 bps apart.

Also provided are multiplex versions of the present base editor systems comprising more than one pair of the first and second fusion proteins, wherein each pair of the fusion proteins binds to a different target genomic region, optionally wherein the first and second cytidine deaminase portions of one pair of fusion proteins are different from the first and second portions of another pair of fusion proteins.

In some embodiments, the base editor system further comprises a nickase that creates a single-stranded DNA break on the unedited or edited strand, wherein the DNA break is no more than about 500 bps, optionally no more than 200 bps, optionally about 10-50 bps, from the cytosine to be edited. The nickase may be, e.g., a ZFP-based nickase, a TALE-based nickase, or a CRISPR-based nickase. In some embodiments, the nickase is a ZFP-based nickase formed by dimerization of a first nickase domain and a second nickase domain fused respectively to two ZFP domains that bind to the target genomic region, wherein one of said nickase domains comprises an inactivating mutation. In certain embodiments, one of the nickase domains is fused to the first or second ZFP-cytidine deaminase fusion protein, and the other nickase domain is fused to a third ZFP domain that binds to a third sequence in the target genomic region. Alternatively, the two nickase domains may be fused respectively to a third ZFP domain that binds a third sequence in the target genomic region and a fourth ZFP domain that binds a fourth sequence in the target genomic region. In particular embodiments, the first and second nickase domains are derived from FokI (e.g., FokI-ELD and FokI-KKR, optionally wherein the inactivating mutation is D450N).

In some embodiments, the base editor system further comprises an inhibitory component of the cytidine deaminase, e.g., a toxin-derived deaminase inhibitor (TDDI) where the cytidine deaminase is a TDD. In certain embodiments, this system comprises a third fusion protein or a third expression construct for expressing the third fusion protein in the cell, wherein the third fusion protein comprises i) a ZFP domain that binds to a third sequence in the target genomic region, and ii) an inhibitory domain for the cytidine deaminase, and binding of the third fusion protein to the target genomic region results in the interaction of the inhibitory domain with, and thereby inhibition of the cytidine deaminase activity of, the dimerized cytidine deaminase portions.

In some embodiments of the inhibitory domain-containing base editor system, the system comprises a third fusion protein or a third expression construct for expressing the third fusion protein in the cell, and a fourth fusion protein or a fourth expression construct for expressing the fourth fusion protein in the cell, wherein the third fusion protein comprises i) a ZFP domain that binds to a third sequence in the target genomic region, and ii) a first dimerization domain; and the fourth fusion protein comprises i) an inhibitory domain for the cytidine deaminase, and ii) a second dimerization domain capable of partnering with the first dimerization domain in the presence of a dimerization-inducing agent; and binding of the third fusion protein to the target genomic region and dimerization of the third and fourth fusion proteins result in the binding of the inhibitory domain to, and thereby inhibition of the cytidine deaminase activity of, the dimerized cytidine deaminase portions.

In some embodiments of the inhibitory domain-containing base editor system, the system comprises a third fusion protein or a third expression construct for expressing the third fusion protein in the cell, and a fourth fusion protein or a fourth expression construct for expressing the fourth fusion protein in the cell, wherein the third fusion protein comprises i) a ZFP domain that binds to a third sequence in the target genomic region, and ii) a first dimerization domain; and the fourth fusion protein comprises i) an inhibitory domain for the cytidine deaminase, and ii) a second dimerization domain capable of partnering with the first dimerization domain in the absence of a dimerization-inhibiting agent; and binding of the third fusion protein to the target genomic region, and dimerization of the third and fourth fusion proteins, result in the binding of the inhibitory domain to, and thereby inhibition of the cytidine deaminase activity of, the dimerized cytidine deaminase portions.

In particular embodiments, the base editor systems described herein comprise both a nickase component and an inhibitory domain component described herein.

Any of the ZFP domains used in the fusion proteins described herein may independently have 2, 3, 4, 5, 6, 7, or 8 zinc fingers.

In some embodiments, the protein components of the present base editor systems are provided to the cells by means of expression cassettes or constructs. Such cassettes or constructs may be provided to the cells on the same or separate expression vectors such as viral vectors. The viral vectors may be, e.g., adeno-associated viral (AAV) vectors, adenoviral vectors, or lentiviral vectors.

In some embodiments of the base editor systems described herein, the cytidine deaminase is a TDD. In certain embodiments, the TDD comprises an amino acid sequence at least 20%, 30%, 40%, 50%, 60%, 70%, 80%, 90%, 92%, 94%, 95%, 96%, 97%, 98%, or 99% identical to any one of SEQ ID NOs: 1-12, or the toxic domain of a TDD comprising said sequence. In particular embodiments, the TDD comprises the amino acid sequence of any one of SEQ ID NOs: 1-12, or the toxic domain of a TDD comprising said sequence. In certain embodiments, the cytidine deaminase is a TDD that comprises an amino acid sequence at least 20%, 30%, 40%, 50%, 60%, 70%, 80%, 90%, 92%, 94%, 95%, 96%, 97%, 98%, or 99% identical to any one of SEQ ID NOs: 13-24. In particular embodiments, the TDD comprises the amino acid sequence of any one of SEQ ID NOs: 13-24.

In some embodiments, the first and second cytidine deaminase portions respectively comprise SEQ ID NOs: 25 and 26, SEQ ID NOs: 27 and 28, SEQ ID NOs: 29 and 30, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 49 and 50, SEQ ID NOs: 51 and 52, SEQ ID NOs: 53 and 54, SEQ ID NOs: 55 and 56, SEQ ID NOs: 57 and 58, SEQ ID NOs: 59 and 60, SEQ ID NOs: 61 and 62, SEQ ID NOs: 63 and 64, SEQ ID NOs: 65 and 66, SEQ ID NOs: 67 and 68, SEQ ID NOs: 69 and 70, SEQ ID NOs: 71 and 72, SEQ ID NOs: 73 and 74, SEQ ID NOs: 75 and 76, SEQ ID NOs: 77 and 78, SEQ ID NOs: 79 and 80, SEQ ID NOs: 81 and 82, or SEQ ID NOs: 83 and 84; or vice versa.

In a related aspect, the present disclosure also provides a fusion protein comprising i) a zinc finger protein (ZFP) domain that binds to a gene (which may be a eukaryotic, e.g., human, gene) and ii) a cytidine deaminase polypeptide or a fragment (e.g., a half domain) thereof, e.g., wherein the cytidine deaminase is a TDD comprising an amino acid sequence at least 90%, 92%, 95%, 96%, 97%, 98%, or 99% identical to any one of SEQ ID NOs: 13-24, optionally wherein the ZFP domain and the cytidine deaminase or fragment thereof are linked by a peptide linker (e.g., comprising the amino acid sequence of any one of SEQ ID NOs: 85-95). In some embodiments, the TDD comprises the amino acid sequence of any one of SEQ ID NOs: 13-24.

In a related aspect, the present disclosure provides a fusion protein comprising i) a zinc finger protein (ZFP) domain that binds to a gene (which may be a eukaryotic, e.g., human, gene), and ii) a cytidine deaminase inhibitory domain, e.g., wherein the cytidine deaminase is a TDD comprising an amino acid sequence at least 90%, 92%, 95%, 96%, 97%, 98%, or 99% identical to any one of SEQ ID NOs: 13-24, optionally wherein the ZFP domain and the inhibitory domain are linked by a peptide linker (e.g., comprising the amino acid sequence of any one of SEQ ID NOs: 85-95). In some embodiments, where the cytidine deaminase is a TDD, the cytidine deaminase inhibitory domain is a TDDI. In some embodiments, the TDD comprises the amino acid sequence of any one of SEQ ID NOs: 13-24.

In a related aspect, the present disclosure provides a fusion protein comprising i) a zinc finger protein (ZFP) domain that binds to a gene (which may be a eukaryotic, e.g., human, gene), and ii) a nickase (e.g., a nickase domain described herein) or a fragment thereof, optionally wherein the ZFP domain and the nickase or fragment thereof are linked by a peptide linker (e.g., comprising the amino acid sequence of any one of SEQ ID NOs: 85-95).

In one aspect, the present disclosure provides a pair of fusion proteins comprising a) a first fusion protein that comprises i) a zinc finger protein (ZFP) domain that binds to a gene (which may be a eukaryotic, e.g., human, gene), and ii) a first dimerization domain, and b) a second fusion protein that comprises i) a cytidine deaminase inhibitory domain, e.g., wherein the cytidine deaminase is a TDD comprising an amino acid sequence at least 90%, 92%, 95%, 96%, 97%, 98%, or 99% identical to any one of SEQ ID NOs: 13-24, and ii) a second dimerization domain, wherein the first and second dimerization domains can dimerize in the presence of a dimerization-inducing agent. In some embodiments, the cytidine deaminase inhibitory domain is a TDDI where the cytidine deaminase is a TDD. In certain embodiments, the TDD comprises the amino acid sequence of any one of SEQ ID NOs: 13-24.

In another aspect, the present disclosure provides a pair of fusion proteins comprising a) a first fusion protein that comprises i) a zinc finger protein (ZFP) domain that binds to a gene (which may be a eukaryotic, e.g., human, gene), and ii) a first dimerization domain, and b) a second fusion protein that comprises i) a cytidine deaminase inhibitory domain, e.g., wherein the cytidine deaminase is a TDD comprising an amino acid sequence at least 90%, 92%, 95%, 96%, 97%, 98%, or 99% identical to any one of SEQ ID NOs: 13-24, and ii) a second dimerization domain, wherein the first and second dimerization domains can dimerize in the absence of a dimerization-inhibiting agent. In some embodiments, the cytidine deaminase inhibitory domain is a TDDI where the cytidine deaminase is a TDD. In certain embodiments, the TDD comprises the amino acid sequence of any one of SEQ ID NOs: 13-24.

In one aspect, the present disclosure provides one or more nucleic acid molecules encoding the fusion protein(s) described herein, as well as expression constructs comprising the nucleic acid molecule(s) and viral vectors comprising the expression construct(s), optionally wherein the viral vectors may be an adeno-associated viral vector, an adenoviral vector, or a lentiviral vector. Also provided is a cell (which may be a eukaryotic cell, e.g., a mammalian cell or a plant cell) comprising a base editor system as described herein, fusion protein(s) as described herein, isolated nucleic acid molecule(s) as described herein, expression construct(s) as described herein, or viral vector(s) as described herein. In some embodiments, the mammalian cell is a human cell, such as a human embryonic stem or a human induced pluripotent stem cell.

In some aspects, the present disclosure provides a method of changing a cytosine to a thymine in a target genomic region in a cell (which may be a eukaryotic cell, e.g., a mammalian or plant cell), comprising delivering a base editor system as described herein to the cell. In some embodiments, the change of the cytosine to the thymine creates a stop codon in the target genomic region. A multiplex format of the system may target more than one genomic region (e.g., 2, 3, 4, or 5 genomic regions). The editing may be performed in vivo, ex vivo, or in vitro.

Also provided are genetically engineered cells (which may be eukaryotic cells, e.g., mammalian cells such as human iPSCs or plant cells) obtained by the present editing methods.

Engineered cells described herein (e.g., engineered human cells), including pharmaceutical compositions comprising the cells and a pharmaceutically acceptable carrier, may be used for treating a patient in need thereof (e.g., a human patient in need thereof) or used in the manufacture of a medicament for treating a patient in need thereof. In some embodiments, the patient has cancer, an autoimmune disorder, an autosomal dominant disease, or a mitochondrial disorder. In some embodiments, the patient has sickle cell disease, hemophilia, cystic fibrosis, phenylketonuria, Tay-Sachs, prion disease, color blindness, a lysosomal storage disease, Friedreich's ataxia, or prostate cancer. Kits and articles of manufacture comprising the cells are also contemplated.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a schematic illustrating a pair of ZFP-TDD fusion proteins for C to T base editing. The rectangles represent DNA-binding zinc fingers in the ZFP domains of the fusion proteins. The arrow shapes above the underlined C nucleotide represent dimerized TDD domains of the fusion proteins. The black lines between the zinc finger domains and the TDD domains represent peptide linkers.

FIG. 2 is a schematic showing ZFP designs for CIITA-targeting ZFP-TDD fusion protein pairs. G2, G5, C6, C8, G10, G11, G14, C15, and C16 are target nucleotides for base editing. Top strand (left to right): SEQ ID NO: 114. Bottom strand (right to left): SEQ ID NO: 115.

FIG. 3 is a schematic illustrating an example of the combined use of the ZFP-TDD base editing system and a CRISPR/Cas-based nickase system.

FIG. 4 is a schematic illustrating an example of a trimeric ZFP-TDD+FokI nickase base editing system.

FIG. 5 is a schematic showing ZFP designs for combined use of CIITA-targeting ZFP-TDD fusion proteins with a ZFP-nickase. Top nucleotide strand (left to right): SEQ ID NO: 116. Bottom nucleotide strand (right to left): SEQ ID NO: 117. Middle amino acid strand (left to right): SEQ ID NO: 118.

FIG. 6 is a table showing the heatmap results of C to T base editing at a human CIITA locus (“site 2”) by a series of ZFP-TDD fusion protein pairs. The degree of editing activity corresponds to the darkness of shading within a cell. Each s2_left_6_ZFP construct is fused to FokI-ELD-D450N. A “yes” in the column titled “Nickase” indicates co-delivery of FokI-KKR.

FIG. 7 is a schematic illustrating a design for inhibition of a TDD with a targeted ZFP-TDDI.

### DETAILED DESCRIPTION OF THE INVENTION

The present disclosure provides systems and methods for base editing, e.g., from cytosine (C) to thymine (T), in cellular DNA such as genomic DNA. The systems entail the use of ZFP-toxin-derived deaminase (TDD) fusion proteins (ZFP-TDDs). By providing precise gene editing in a cellular context, the present systems and methods can be used for the prevention and/or treatment of numerous diseases. It is contemplated that these systems and methods will be particularly useful for cell-based therapies that require the simultaneous knock-out of multiple human genes.

The present systems and methods can convert targeted C:G base pairs to T:A base pairs. In some embodiments, the base editing systems may also include proteins (e.g., UGI) that increase the stability of the conversion, and/or endonucleases that nick the DNA near the targeted base so as to stimulate DNA repair in the edited region and to promote the correction of the G nucleotide on the opposite strand to A, forming the edited T:A base pair.

The present systems and methods are advantageous in part due to the compact size of the ZFP domains in the fusion proteins. In comparison, the large physical size of a TALE and the long C-terminal TALE linker may limit how small the base editing window can be, as well as design density. The size and highly repetitive nature of engineered TALEs also make it challenging to deliver TALE-based base editors to human cells using common viral vectors. The present ZFP-derived base editing systems circumvent these problems. For instance, the compactness of these ZFP-derived systems may allow for packaging within a single AAV vector, in contrast to TALE base editor systems (e.g., TALE-TDDs) or CRISPR/Cas base editor systems. In addition, due to the small size of the fusion proteins herein, it is possible to include a nickase in the editing system so as to allow the generation of a DNA nick near the edited base and thereby facilitate the DNA repair machinery to change the base opposite the edited C from G to a corresponding A, forming the correct T:A base pair. The inclusion of a nickase may greatly increase the base editing efficiency.

### I. Zinc-Finger Fusion Proteins

Provided are fusion proteins that contain a DNA-binding zinc finger protein (ZFP) domain fused to a base editor domain (e.g., a cytidine deaminase domain, which may be a TDD such as one described herein) or a fragment thereof, a cytidine deaminase inhibitor (e.g., a TDDI) domain, and/or a nickase domain (e.g., a FokI domain). As used herein, a “fusion protein” refers to a polypeptide where heterologous functional domains (i.e., functional domains that are not naturally present in the same protein in nature) are covalently linked (e.g., through peptidyl bonds). These fusion proteins, which can be recombinantly made, are components of the present base editor systems. In some embodiments, a ZFP fusion protein herein comprises a cytidine deaminase domain (e.g., derived from a TDD as described herein) and additionally a nickase domain and/or a UGI domain.

Other formats of the present systems also are contemplated herein. For example, instead of peptidyl links, two functional domains may be brought together by noncovalent bonds. In some embodiments, two functional domains (e.g., a ZFP domain and a cytidine deaminase inhibitor domain; or a ZFP domain and a nickase domain) each are fused to a dimerization partner (e.g., leucine zipper and those described further herein), such that the two functional domains are brought together through interaction of the dimerization partners. In certain embodiments, the dimerization of these domains may be controlled by the presence or absence of a specific agent (e.g., a small molecule or peptide). It is contemplated that such formats may substitute for fusion proteins in any aspect of the present invention.

Each component of the present base editor systems is further described in detail below.

### A. Base Editors

The ZFP-cytidine deaminase fusion proteins of the present disclosure comprise a cytidine deaminase domain or a fragment thereof in addition to a ZFP domain. The term “deaminase” or “deaminase domain,” as used herein, refers to a protein that catalyzes a nucleoside (e.g., cytidine, adenosine, deoxycytidine, or deoxyadenosine) deamination reaction in the context of a free base, RNA, or DNA. A cytidine deaminase domain, for example, may catalyze the deamination of cytosine to uracil, wherein the uracil is replaced by a thymine base during DNA replication or repair. The deaminase domain may be naturally-occurring or may be engineered. In some embodiments, a cytidine deaminase of the present disclosure operates on double-stranded DNA.

In some embodiments, the cytidine deaminase is derived from a toxin that may be, e.g., from a prokaryotic or eukaryotic organism. In certain embodiments, the organism may be bacteria or fungus. Such a cytidine deaminase is referred to herein as a toxin-derived deaminase (TDD). As used herein, a cytidine deaminase “derived from” a toxin may refer to a cytidine deaminase that is the same as the naturally occurring toxin or is a modified version of the toxin that retains deaminase activity.

In certain embodiments, the TDD may comprise, for example, an amino acid sequence selected from SEQ ID NO: 1 (“TDD20”), SEQ ID NO: 2 (“TDD21”), SEQ ID NO: 3 (“TDD22”), SEQ ID NO: 4 (“TDD23”), SEQ ID NO: 5 (“TDD24”), SEQ ID NO: 6 (“TDD25”), SEQ ID NO: 7 (“TDD26”), SEQ ID NO: 8 (“TDD27”), SEQ ID NO: 9 (“TDD28”), SEQ ID NO: 10 (“TDD29”), SEQ ID NO: 11 (“TDD30”), and SEQ ID NO: 12 (“TDD31”), or a part of said amino acid sequence that is capable of cytidine deaminase activity (e.g., a “toxic domain”). These amino acid sequences are shown below:

TDD20 (NCBI Accession No. WP 212528010.1)
(SEQ ID NO: 1)
MVGLVAGLALASPMPAVAAVAARPAASSGPAVTGVVMSSKSHTNTRTMTLNGKSVGVYKASG
TQLPAATSGSATLQARGTATVSGNQPPAVTAAQLPKASIAGTPLWAQRTSTLSGPSSVTGAV
ASQSLAKQLGVTGVVYSVAGSGGSGSVRVGLDYAAFKDAYGANFGSRLELYTLPACALTTPQ
LAKCRVRTPVTGAVNDPAADTLSGVVKISGADTAASSGAAYTGGGTVSDGNYVIGTASAVSA
SSPSSGIVLAASSGAGEEGSATGNYAATKLSPAGSWTAGGSEGDFTYNYPITVPSSSTSLTP
KVELDYDSGSVDGKTSMTNAQASMVGDGWTDPSENYITQTYVPCSDSPEGTASPTSTQDMCY
DGEILSISLNGSSTTIVDDNGTFKLQNDNGAVISHVADSNKGQNTYNTDYWTVTERDGTEYY
FGLNELPGYTSTGQTNSVDWEPVYAAHSGDPCWNATWADSVCNMAYEWHLDYVTDTHGDAMS
YYYKQDTNYYGANNGASEKEYVRDSYLSEIDYGYTTASGAYGIVPDKVSFTTVNRCVASTCD
APSSSMSATTAASEYPDVPTDLICASGATCTSYSPSFFSTVMLTTITTSQYSLSASKQVDVD
SYALAQDFPATGDNTSGTLWLESITHTGDDTSAGGSSSSISEPSVSFSGTDLPNRWDVETYP
GLYRWRIADVTSELGSKVGVTYEIPDTCAASTLDAPTATPSSNTTSCYPVYWTPDGYSAAIE
DWFIKYAVREVTVTDETGGAAEEVTQYSYANPAWHYDDEPAVQAKDRTYGEFRGYEQVNTVQ
GNGTSDAMDKSVTRYYQGMYGDYLSPTSTSTTTVPDTLGGVHNDYAALAGQPLETLTYFGDT
STVDKATVDSYWVSSATASQTFTGLPATTAQMTGPAEAYTEQLVTDSSTTSAWDYTETDDAY
DATTTDADFGLKLYEYSHAWTSSGTADTDDTDTDYSHCTSYTYAPANTTLNIVGLSLATTEA
SVACSGFTESSIPSVPSTSTSLGAPASFTQDQVVSATLNFYDQNGSFVTTGIAPQTTTPTVG
NLTETATATGYAPGTFTYQMASESTFDNYGRAEDTYDADGYKTITSYTVTDGITTAESVENA
LNQTTSETFDPARALVLTSTDINGVVTTKQYDALGRVTAVWGYSRATSTAANYLYSYTESKT
GLSGSITEKLNDLADYTETATILDSLGRTRETQANTPAGGSLVTDTIYNSLGQVSATYNNWW
DSTDLPSIALASSFDIDQIYNWDQYVYDGLGNKVEDESMTYDDQVYSTSYTVYSGDETTTFP
PAGGTVKSTGTDPLGRTSSVSTYSTRPTVTIPSNVNTTPFYVSGGVALTTSYTYDGHGNQAV
TTDPDGQKWTSVFDLLGRVTEKEDPTAGNSFTVYDADGNVLQTEDARSEYVSYTYDKLGRKT
AEYASDDVAADQAKAGAEVAAWVYDDSNGLAAISAYAKGHVTTEYSYSGGATYTLQYEGFNV
FGESLEEAYTFPSTVTGLAGTYYIGHSYTANLGLPASTIYPTTTGPLATETVGYSYTGALEL
PNGVNGTGQYSQSTAYDAWGRVSSETIGSTAATGEATLTNTYDPHTGALTEQLVTNATGNID
ETTYAYDLYGNITKQLDERENTAADTETQCYQYNGLDQLSTAWTATDACATVPSQNSFSTVG
DALGTASAYWDSWTYDNEGNRQTQDQHSLTSSAQDQITTDTISSTNNEISGTTTSVNSAQTA
STTYGYDLAGNTDSRTTTADGTQTLSWNNEGQLYNISSTKNGTSAYVYDADGNLLTETDSAT
NSTTLYLPGEQITYNTGNGATSVNRYYALADGTAVRTGTGNNYDFELSDQHHTNDLYLDYTA
QTPTWRQFDPFGNARGTAVTWVDNRTFLNDVTDTETGLTDIGARWYDAAIGRFESLDPVFEA
TDTLALGGYAYTDGNPITSSDPTGQMIEGESGYGTANDSQCDLSCATNLANNVTQKNQESIE
AEHSHSGGLGTKVLALLCGVCAAVPEMYDQGAKIDPTNPAQGMINNADGETAIQQGIATDNP
QELQNGFVQLGLTLAPGLEEAGALDDSSDLLMGACLGGESFTADTPVLTATGKQVAISTLKV
GDKVEATNTSTGKTEPEKVSAVLVHHDTDLYDLTVRVGGKPSVIHTTSNHLYWVPGTGDKNH
GGKWVTAAQLKPGTHLRTPDNATAVTVVAGATPAKPDGWMWDLTIPGDNDHDFYVVIGGTAV
LVHNINGCGPSAVQQLLSANGEPGKTAGVLDLNGELTSLVSGKGELPNYAASGHVEGQAAMM
MRAEKATSATLYIDNPNGICGYCRSQIATLLPEGATLEVVTPLGTVEPTARWSSSKVFTGNE
RYPKGWVE
TDD21 (NCBI Accession No. WP 166233360.1)
(SEQ ID NO: 2)
MAAPGACTRTRLVTRSNSPCPPRAQASAGRGLAAPGTGAVASQSRGRRPVMDQTPPEARYVA
AGHIEGKAALQVRAAASSGGTIHHNNPFGTCIYCQQQVPTLLPDGARLTAEPPAQARPRSAW
WSPPGPVTMVGNVADPASHPVATAQ
TDD22
(NCBI Accession No. MBK7217240.1)
(SEQ ID NO: 3)
MKHGRDSWYPIASDADLLVKTLLFASVADASFDKASDGEEYLGHLPTRTRTRDKTHGLLVLD
DEPPIPVVSGQNGPAATIPRGSRGFDAITQTHVEGHVAALMVNHGWTRATLYINNVPCPSCD
RLLPRMLPPGARLNVIGPNGFQKLYVGGRW
TDD23 (NCBI Accession No. RTL35118.1)
(SEQ ID NO: 4)
MPAEVTEKSQPAPPAQAESGMSQAAQSAWAPGSNYFRLGAVCNDTFNQSLSTQYDGLPQCAI
AGIGEGLKKLSAIGEVGVGVAADVASVVVADLQIGYEAITYPSLAEGNSDRLAHKLRELQTK
YEPLTQSVKQGYEGVQHDLNDIGAQSNNDSAAYARLVTDPAQSIVKAIDKFTGQTPAEQVKD
IFDQGTQIAVAAALGKAAALASGSCRLEEIAAKFDALFSKMEETKQAFGTQLEHCIASGKKV
QSSDYIRLSKAGDQDGLSRPADKISESCNSVDRSYFERLPQRMRDSDKTAGILVADGQELDV
LSGRKGPAQSMPAQSRGFDLITKTHAEGHAIAHMIQHGIKEAKIYINNNEICSSCEKLIPRM
LPPGSKLEVHMPSGAVRNFVGEPR
TDD24 (NCBI Accession No. WP_216577045.1)
(SEQ ID NO: 5)
RGSVRAVTDQTGTVTDRYSYSLYGERTHDTGDGDHIFGYCGRDGVITERDGLLYMRARYYSP
ELKRFISADSVIGNLSSTPSLNRYAYVNGNPVTQIDPFGLSAEPGTKKAKKNINWREAVHDA
LDIAGSITGYGDICDAVNAVLYLTEGDYINAGLSALSIIPGADAFTKTGKASKVIGNGIVES
GLKHLDNVADGIKKLVKKADDAIDLEKHLPKKLLTEEAEAFAKKADKVADDVVEDAAKAIKG
GSSSINLPEYDGKTTHGVLVLDDGTQVPFSSGNANPNYKNYIPASHVEGKSAIYMRENGINN
GTVFHNNTDGTCPYCDKMLPTLLEEGSTLTVVPPANANAPKPSWVDTVKTYIGNDKIPKKPK
TDD25
(NCBI Accession No. CP070896.1; bp 1412377-1412931)
(SEQ ID NO: 6)
FFYLKTAQRRAIIFLIEYENSPFVAYLSTPPEGSGQKNNGNTTTPDFYVSPDGHPIPRAAYN
LPEYDGSTTHGVLITSDGSEISFTSGGGDPRYTNYANNGHVEQKSSLYMRDNNISSGVVYHN
NTEGTCGFCNTMTSTFLPEGATLTVVPPSNAVATNSKAVDVPKTYTGNSKDPKISSRYTGN
TDD26 (NCBI Accession No. MBV9870847.1)
(SEQ ID NO: 7)
MARIQNGVNLHDGSTMDVPVTGVANVPTSGVTAVAISVTTQENNPQGGTLMVYPKGGTPSDP
ELPYPANGKFQSTTIFVQPDSVSGKITISNTNGDGYGVIVAVDVEGYFAANGSQNGFFPMIG
ERILFPGGGGVVDNTSIDATNPVTVKVAGVGGVPQNAASVAINIEATNTHFGAIYAYPSDAN
PAPSGVSELTMNTGVNADSLVQVSTTTSNSITIASTAAATIRLDVEGYTISGSNSPNAGAFV
PLSDPQTVVPAQVVTSGNRIFQKVTNQDPGTAKDNIPSAASAVVGVLSAGSATAAGTARIWP
SRQPAPGISQLEFGAAGAWSNLMIVPIGNEGQVNVRADLTGGTGSVAMTFVAIGYYTEAAPL
PDTVSDPTPYLGNENYWTYARPTSVGPQSTAKVNVSNGNLVVSATDATPVPGHGHLDFNLKR
TYNSLDNPGGTGVVDALEGLAGGFTKGTIGTGWTLSLGGFGTGVDSGFDTGGLTSLITPDWV
AGTQQTVTLPTGQLILVDQDGTRHVFTADGAPLGAASNLLAKQGNIQTTGLGRTLGSLFKVT
AAGNGSTLSQLLQITGNDKLDGSFCVYQPYNPPAGVHLSLFSVAYIQTVGTNDCVGGSNPVT
VAYIAERPDGLRFVYSSGGQLLDEVDRAGDDLRFAYEGPLTAAVNGTTIANYGSLQHLPLVG
DGSPINPSLKTVWEANVPGCTDGATGQFTGNSKCRSIGITYSERSEAGCTTTAPQTAVCVTD
AAGRMTKYVLDGAPILSTPAHLVTVINPDDSTVNYTYGCNGGAVNQLCSVTDPNGGVATAIS
YLGTSAYSDSIDNGPLVSQVTDRRGIKTIFNYNVGHMAASAGAQFRGLSSTTEAVTQVALGD
NCDLNVTASAGCRVSQYTAIDNRGRVGEIDRGTLSSSSYCSGQPAHPVDGAGICAQQVTTFG
WDTRHLPIPSFAAKGSATAKATGGILPMGGGIKYSYVVTGVEGGGETPPSAVATTSSIATSS
SGSVTLKWNAEVGASAYRIYGRTYNGTDMSTIKLIAEVPSTVNELNQPGSQFTWTDDGRTTP
SNALPSTDTTQQVLCSAFDSNAVDNLLCRTTQLALNDNDTGQANLVFAADRATNYSYGLDGQ
PVLSDVVMPGSGGDLKTTYGYGYQYWDSAGDVVATGTHPTGSGNVSTPAFSSSDTLFTISDK
TAMLPPRGNAAGGSYAPYETIYDSDDLSKVTDQQSQVTTYYPPNTVAGSGDPAGLSVCSTDG
GTSGGKAGKPTANTGLMCEVDTPTPARASDAGGVSNCQAVDLAFAAVCTTYTYNGWGEKTSM
TTPLAHTDDLGSANKFAAYHYVYYADTDTDVATATPSGGWLKAVVDPIAKKDDTVTDLSGLT
QHFVVFGYDAAGNTVLTWDRYAVQRNTAADIAKGGTHDGIELSSYTPSESGGAPASGNQPGY
SYQTYGSANYGASSSRDLFAKPWRYVVKSATAETSADPTVAPFEAPAPNYTTAPNETTVIVV
DGNGNPTSITDPLGNATVNTYDAIDELKTSSTHAKTAGEKTTYVYDGDGNPITITSPNGQSG
VAITTGNSPGFSTHIVYNALDLPIQTVTPRLANGDAGANLPAPFKGDKCSQGIGGYDALYDC
QTVTVYDGFGEPVETIDGNNHVVYSFYDAAGRKIARSVSYGQPTSALNQADLNSQGTRSATP
TGADYLNSAWVYDQDGNITKTCTPRDFDPNGGNAKAADNSTPGTSAPSTSAKLDTLTGACSN
NVPLYGSTSTYGNADETTSVSKYRAPGSPATPDGTTVPSGTKALTTTSTYDADGNTVNVLDP
AGTAGVAPNQASHVAMKYTYDSLDRKVCQSMPRLTSGLPAVPSDCTRTATQSAYTVTHYNDS
GDVESVEAPKGDSASSAGGNLSRRSDTMYDLDHRVVATIAGATSADPFSTYLADKANQQDLR
TGYYYDAGGNLIQQFDPRAYARANALKAPATRTDSNGPDPDYSVIIRYDGNHHPTKILSPRA
DSTLAHQSQPEGSQCPTSNAPTGSDVYKAFPTSPTTSVCETDVTYDRNGNRWTILLPTQVSG
GQQPTVSYTYTYDDLLSSTATPSPTGTGSSTTTTYYDGDAQPVKTVDPDTKTTWTKYTGNEL
PYDVADADPNAIAPAKLLHHAITAYDANGATITSAGLVSNTGGWPNIGGSDGGGSWQVTTTA
INSDGTTQTSTNTSRSMNGDTTTYDYDLNGNPTKIYSPKGSPPPASPTTVNTYTADNLLATS
ARSVTTSDSRTTSYTYDPAGRKEKQAIADGSLGAGGNLQFTYYPTDEPASQTGRDPSAAGNT
TTTGTPDTFANGSGTVSFGYDEAGNQSTVNDSANYGGGSGSYGSSITNYYTLDSLLAETDEG
PNADYLRYDGSGSVTMRTSQIGATRNQWIHFTNGPSGLPTAADTNQASGHWSFNYDSAGLLT
DATYPDGEALHNNYYTGANNAATNYDSEMLKDTTLTSGVNTLQKFAYSYDGLDRIKTQNDQR
AGGKLYTFSYDPQGRICQFDDGSATASTRNITWDADGNRTAFGNVGEENGCQGTGSNLTGSS
TTTNYNLDDTIKTIGATSVAYDAAGRLTNDGVNNYCYDGFDQTELVGAASDTSCTTSVGTTY
AYDGLSRQASSTKNSNTSKYLYDGTSSSLLTDSRSQGTAHGAINYLLSPTDAPLAIADDTNS
GNITQYLTDDGAGNIGAVTDATTHLALCNPRYDPFGTLLPPAAGVPAPPASCPAYVGTLYYG
ANRRDANTGNYQDGARTYNPTLDAFTTADSYGPGDGSDDQSIGVDPLTANTYTYVNGDPINY
DDPTGHYQAADTGAKPAAHGAVAPTYAQIVWELKYEEQQREEQIVHDLMSQVNSWSGCWSCI
GSNLSQAGHDIASTATGVYNGAKNLASSCASGHWGTCGKGLGAIAIGVAAAFACPETAGATC
ALLAAGAAGAGGGALTCNGDSTANCAGKGALIGGLTFGIVKGGGAIRGVAGGAATNADAQAL
LGSIRQAGKTAGVLNIDGDLMPLVSRKSSLPNYAASGHVEGQAALIMRERGVSSAELLIDNP
NGICSYCTSQVPTLLPEGAQLMVRPPLGTVPTQWWFNGRTFLGNAANPKPSPW
TDD27
(SEQ ID NO: 8)
MQGFVNAGELAIGDELLDSSGNILFVEKFDVELTEKPTKVYNFQVEDFHTYYVGENGVWVHN
ASSEYGEVPELPEFNGKKTEGVFRTADGKEIKFESGGSTEYKNPSASHAEGKAAIYMRENGI
KEGTVFHNNPNGTCNYCDKGLATLLPEGARLTVVPPIGAVAPNKYWVDVPKTYTGNGNLPSM
K
TDD28
(SEQ ID NO: 9)
MKKRLISFMLLITVISCVGFNTDYFKNLLTLISQAAKEVIYEETESYDEETSDSAVDRLEQE
LEKLDVRNFFEDESETESGSSSINLPEYDGKTTHGVLVLDDGTQVPFSSGNANPNYKNYIPA
SHVEGKSAIYMRENGINNGTVFHNNTDGTCPYCDKMLPTLLDEGSTLTVVPPTNASAPKPSW
VDTVKTYIGNDKIPKKPK
TDD29
(SEQ ID NO: 10)
MRKKILKFILIQLMLVSFIFTNCGSMLKEMCTFAEQISSNEENTDKASIISCGDFNSDGKIN
VFDLMRSKQHVLNKEEQPSGLADINADGAFNILDSLILQDYILLRDTKIKDKVNVPPSDNIP
DVSSDDSYRYVIFSEDKETPIQINSQLTTINGKIATNNNFEGNRSINGDGKIIENANISMVY
MDDKIVNTYFSNPEEYYEFVIENDDEANKIIDTPTISYDNILIEKSVTINSCMMAMNDIDIK
SNTDNTGNTVIYSKYGDINIDCSNINLVGLIYAPFGTVKLSAQNVQIDGIIIAKNVIIDSEQ
KINLNPNNSIADFIGVDTEKMVIPYRDWCYIDKPDDAVLPEIVEEQIYEDPLSVDDAWLEDK
YGSLIVDEYSAQMKKELVECLYICISERISNLSELIELFSFAAQGNAKDYVKLKTSLKNGKK
DVSLTIGDYDISVEKEKSDKVIYGPMPYNPFVFRYYLTPNKTSDDSDNTDEPQEEPKKFKIN
DIGLFFELAAAGVSTAVVNVATTAGTAGTAVIGAGTAVIGAGTAAAATAINTVTTTVTKITT
KAEQRIEQAVQNGKDSIVNIKNEVETSIKDTLSDIADVDLFGMQYVPLINGFGYNIGSVRLN
FIKEVGSWTSQTILNGEPINIDKIKTVVDLDTLNNINSISPQIYKDIASNIMNDKDLEKTYK
IIDCINEKTDEFKLAYEQLKDIEQAYEYANIDKDKIDIPDFDGKITWGILVLEDGTCITFSS
GNANPMFNHYIPASHAEGKAAIYMRQKGIKHGVIFHNNTDGTCPYCNTMLPTLLEENSTLIV
VPPINAVAKKRGWIDKIKIYTGNNKIPKIN
TDD30 (NCBI Accession No. MBR1615955.1)
(SEQ ID NO: 11)
MGNYESDKSRLTPYLFIVEVDGIQTMRFQKCEGLEAETEVFEYQEGGGTVHNFKGRSRFPNI
VLEKGIVDSDDLWKWYNKTAEGNVERQSGSVVLCNLQGEEIKRWNFFRAFPCRWVGPALNAM
DRRTFAVERIEIAHEWLEVDGGDEEEENLTSNNEKKKNANKQKIELPPYDGKTTYGVLILDD
GKQYSFNSGKPAPIYRNYIPASHVEGKAAIYMRENKIQSGTVYHNNTDGTCPYCDKMLPTLL
EKDSTLKVVPPQNATSSKKGWITNEKIYIGNDKIPKTAR
TDD31
(SEQ ID NO: 12)
DMEAGLGESGRAHIALPEYDGGTTHGVLVLDDGTQIPLSSGKGDPPGTNYAASGHVEGKAAL
IMRENGITSATLYINNPNGTCGYCDSQLPTLLPEGATLTVVPPANAVAPKSRWIDYPKTYIG
NSNDPKPWPRYKGN

In some embodiments, said sequences do not include a signal sequence, if present.

In some embodiments, the cytidine deaminase may comprise the toxic domain of a TDD. Examples of toxic domains for TDD20-31 are SEQ ID NO: 13 (TDD20), SEQ ID NO: 14 (TDD21), SEQ ID NO: 15 (TDD22), SEQ ID NO: 16 (TDD23), SEQ ID NO: 17 (TDD24), SEQ ID NO: 18 (TDD25), SEQ ID NO: 19 (TDD26), SEQ ID NO: 20 (TDD27), SEQ ID NO: 21 (TDD28), SEQ ID NO: 22 (TDD29), SEQ ID NO: 23 (TDD30), and SEQ ID NO: 24 (TDD31), e.g., as shown in Table 3. As used herein, unless specified otherwise, the term “TDD” refers to the TDD toxic domain.

In particular embodiments, the cytidine deaminase domain (e.g., derived from a TDD described herein) is a “split enzyme” comprised of first and second “half domains” or “splits” that lack cytidine deaminase activity alone, but dimerize to form an active cytidine deaminase. As used herein, half domains that are “inactive” or “lack cytidine deaminase activity” may be half domains that i) lack any cytidine deaminase activity (e.g., any detectable cytidine deaminase activity), ii) lack specific cytidine deaminase activity, or iii) lack significant cytidine deaminase activity (i.e., on-target base editing activity of 1%, 2%, 3%, 4%, 5%, 6%, 7%, 8%, 9%, or 10% or more, which in particular embodiments may be 10% or more). For example, assembly of the active cytidine deaminase may be driven by the binding of half domain-linked zinc finger proteins to DNA targets in proximity to each other such that the half domains are positioned to allow assembly of a functional cytidine deaminase.

It is understood that the “half domain” pairs described herein may refer to any pair of cytidine deaminase polypeptide sequences that separately have no cytidine deaminase activity, but together form a functional cytidine deaminase domain (either wild-type or a variant discussed herein). In some embodiments, the toxic domains of TDD20-TDD31 are split into half domains at the residues indicated in Table 3. In certain embodiments, TDD half domain pairs may comprise the amino acid sequences of SEQ ID NOs: 25 and 26, SEQ ID NOs: 27 and 28, SEQ ID NOs: 29 and 30, SEQ ID NOs: 31 and 32, SEQ ID NOs: 33 and 34, SEQ ID NOs: 35 and 36, SEQ ID NOs: 37 and 38, SEQ ID NOs: 39 and 40, SEQ ID NOs: 41 and 42, SEQ ID NOs: 43 and 44, SEQ ID NOs: 45 and 46, SEQ ID NOs: 47 and 48, SEQ ID NOs: 49 and 50, SEQ ID NOs: 51 and 52, SEQ ID NOs: 53 and 54, SEQ ID NOs: 55 and 56, SEQ ID NOs: 57 and 58, SEQ ID NOs: 59 and 60, SEQ ID NOs: 61 and 62, SEQ ID NOs: 63 and 64, SEQ ID NOs: 65 and 66, SEQ ID NOs: 67 and 68, SEQ ID NOs: 69 and 70, SEQ ID NOs: 71 and 72, SEQ ID NOs: 73 and 74, SEQ ID NOs: 75 and 76, SEQ ID NOs: 77 and 78, SEQ ID NOs: 79 and 80, SEQ ID NOs: 81 and 82, or SEQ ID NOs: 83 and 84.

Where the present disclosure refers to a cytidine deaminase (e.g., a TDD as described herein), it is contemplated that other cytidine deaminases can be used in the fusion proteins and cell editing systems described herein. The cytidine deaminase can comprise wild-type or evolved domains. In certain embodiments, the cytidine deaminase may be, e.g., apolipoprotein B mRNA-editing complex 1 (APOBEC1) domain or an Activation Induced Deaminase (AID).

The present disclosure also provides other potential cytidine deaminases. Such cytidine deaminases may be used, e.g., in the fusion proteins and cell editing systems described herein. In some embodiments, the cytidine deaminases are functional analogs of a TDD described herein. A functional analog of a TDD is a molecule having the same or substantially the same biological function as said TDD (i.e., cytidine deaminase function). For example, the functional analog may be an isoform or a variant of the TDD, e.g., containing a portion of the TDD with or without additional amino acid residues and/or containing mutations relative to the TDD (such as a variant with at least 70%, 75%, 80%, 85%, 90%, 95%, 96%, 97%, 98%, or 99% sequence identity to the TDD (e.g., a TDD comprising the amino acid sequence of any one of SEQ ID NOs: 1-12) or its toxic domain (e.g., a toxic domain comprising the amino acid sequence of any one of SEQ ID NOs: 13-24)). In certain embodiments, the functional analogs are orthologs of a TDD described herein. In certain embodiments, a TDD ortholog may comprise an amino acid sequence at least 20%, 25%, 30%, 35%, 40%, 45%, 50%, 55%, 60%, 65%, 70%, 75%, 80%, 85%, 90%, 95%, 96%, 97%, 98%, or 99% identical to the amino acid sequence of said TDD (e.g., a TDD comprising the amino acid sequence of any one of SEQ ID NOs: 1-12). In certain embodiments, a TDD ortholog may comprise a toxic domain with an amino acid sequence that is at least 20%, 25%, 30%, 35%, 40%, 45%, 50%, 55%, 60%, 65%, 70%, 75%, 80%, 85%, 90%, 95%, 96%, 97%, 98%, or 99% identical to the amino acid sequence of the toxic domain of a TDD described herein (e.g., a toxic domain comprising the amino acid sequence of any one of SEQ ID NOs: 13-24).

In certain embodiments, a cytidine deaminase described herein may target a cytidine in an AC sequence, a TC sequence, a GC sequence, a CC sequence, an AAC sequence, a TAC sequence, a GAC sequence, a CAC sequence, an ATC sequence, a TTC sequence, a GTC sequence, a CTC sequence, an AGC sequence, a TGC sequence, a GGC sequence, a CGC sequence, an ACC sequence, a TCC sequence, a GCC sequence, a CCC sequence, or any combination thereof. In certain embodiments, a cytidine deaminase described herein has increased efficiency and/or activity compared to DddA. In some embodiments, the increased efficiency or activity may be, e.g., at any one or combination of the above target sequences.

The term “percent identical” in the context of amino acid or nucleotide sequences refers to the percent of residues in two sequences that are the same when aligned for maximum correspondence. The percent identity of two amino acid sequences (or of two nucleic acid sequences) may be obtained by, e.g., BLAST® using default parameters (available at the U.S. National Library of Medicine's National Center for Biotechnology Information website). In some embodiments, the length of a reference sequence aligned for comparison purposes is at least 30%, (e.g., at least 40, 50, 60, 70, 80, or 90%, or 100%) of the reference sequence.

It is also contemplated that adenine deaminases (e.g., TadA) may be used in the fusion proteins and cell editing systems described herein for conversion of A:T base pairs to G:C base pairs. In certain embodiments, a TDD may be mutated at residues that form the nucleotide pocket to allow the enzyme to act as an adenine deaminase, and/or to reduce TC sequence bias within the base editing window.

### B. Zinc Finger Protein Domains

The fusion proteins described herein (such as ZFP-cytidine deaminase (e.g., ZFP-TDD), ZFP-cytidine deaminase inhibitor (e.g., ZFP-TDDI), or ZFP-nickase fusion proteins) comprise zinc finger protein (ZFP) domains. A “zinc finger protein” or “ZFP” refers to a protein having DNA-binding domains that are stabilized by zinc. ZFPs bind to DNA in a sequence-specific manner. The individual DNA-binding domains are referred to as “fingers.” A ZFP has at least one finger, and each finger binds from two to four base pairs of nucleotides, typically three or four base pairs of DNA (contiguous or noncontiguous). Each zinc finger typically comprises approximately 30 amino acids and chelates zinc. An engineered ZFP can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers that bind the particular triplet or quadruplet sequence. See, e.g., ZFP design methods described in detail in U.S. Pat. Nos. 5,789,538; 5,925,523; 6,007,988; 6,013,453; 6,140,081; 6,200,759; 6,453,242; 6,534,261; 6,979,539; and 8,586,526; and International Pat. Pubs. WO 95/19431; WO 96/06166; WO 98/53057; WO 98/53058; WO 98/53059; WO 98/53060; WO 98/54311; WO 00/27878; WO 01/60970; WO 01/88197; WO 02/016536; WO 02/099084; and WO 03/016496.

The ZFP domain of the present ZFP fusion proteins may include at least three (e.g., four, five, six, seven, eight, nine, ten, eleven, twelve, thirteen, or more) zinc fingers. Individual zinc fingers are typically spaced at three base pair intervals when bound to DNA. unless they are connected by engineered linkers capable of skipping one or more bases (see, e.g., Paschon et al., Nat Commun. (2019) 10:1133 and U.S. Pat. Nos. 8,772,453; 9,163,245; 9,394,531; and 9,982,245). A ZFP domain having three fingers typically recognizes a target site that includes 9 or 12 nucleotides. A ZFP domain having four fingers typically recognizes a target site that includes 12 to 15 nucleotides. A ZFP domain having five fingers typically recognizes a target site that includes 15 to 18 nucleotides. A ZFP domain having six fingers can recognize target sites that include 18 to 21 nucleotides.

The target specificity of the ZFP domain may be improved by mutations to the ZFP backbone as described in, e.g., U.S. Pat. Pub. 2018/0087072. The mutations include those made to residues in the ZFP backbone that can interact non-specifically with phosphates on the DNA backbone but are not involved in nucleotide target specificity. In some embodiments, these mutations comprise mutating a cationic amino acid residue to a neutral or anionic amino acid residue. In some embodiments, these mutations comprise mutating a polar amino acid residue to a neutral or non-polar amino acid residue. In further embodiments, mutations are made at positions (−4), (−5), (−9) and/or (−14) relative to the DNA-binding helix. In some embodiments, a zinc finger may comprise one or more mutations at positions (−4), (−5), (−9) and/or (−14). In further embodiments, one or more zinc fingers in a multi-finger ZFP domain may comprise mutations at positions (−4), (−5), (−9) and/or (−14). In some embodiments, the amino acids at positions (−4), (−5), (−9) and/or (−14) (e.g., an arginine (R) or lysine (K)) are mutated to an alanine (A), leucine (L), Ser (S), Asp (N), Glu (E), Tyr (Y), and/or glutamine (Q). In some embodiments, the R residue at position (−4) is mutated to Q.

Alternatively, the DNA-binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. Nos. 5,420,032 and 6,833,252; Belfort et al., Nucleic Acids Res. (1997) 25:3379-88; Dujon et al., Gene (1989) 82:115-8; Perler et al., Nucleic Acids Res. (1994) 22:1125-7; Jasin, Trends Genet. (1996) 12:224-8; Gimble et al., J Mol Biol. (1996) 263:163-80; Argast et al., J Mol Biol. (1998) 280:345-53; and the New England Biolabs catalogue. In addition, the DNA-binding specificity of homing endonucleases and meganucleases can be engineered to bind non-natural target sites. See, for example, Chevalier et al., Mol Cell (2002) 10:895-905; Epinat et al., Nucleic Acids Res. (2003) 31:2952-62; Ashworth et al., Nature (2006) 441:656-59; Paques et al., Current Gene Therapy (2007) 7:49-66; and U.S. Pat. Pub. 2007/0117128.

In some embodiments, the present ZFP fusion proteins comprise one or more zinc finger domains. The domains may be linked together via an extendable flexible linker such that, for example, one domain comprises one or more (e.g., 3, 4, 5, or 6) zinc fingers and another domain comprises additional one or more (e.g., 3, 4, 5, or 6) zinc fingers. In some embodiments, the linker is a standard inter-finger linker such that the finger array comprises one DNA-binding domain comprising 8, 9, 10, 11 or 12 or more fingers. In other embodiments, the linker is an atypical linker such as a flexible linker. For example, two ZFP domains may be linked to a cytidine deaminase, inhibitor, or nickase domain (“domain”) such as those described herein in the configuration (from N terminus to C terminus) ZFP-ZFP-domain, domain-ZFP-ZFP, ZFP-domain-ZFP, or ZFP-domain-ZFP-domain (two ZFP-domain fusion proteins are fused together via a linker).

In some embodiments, the ZFP fusion proteins are “two-handed,” i.e., they contain two zinc finger clusters (two ZFP domains) separated by intervening amino acids so that the two ZFP domains bind to two discontinuous target sites. An example of a two-handed type of zinc finger binding protein is SIP1, where a cluster of four zinc fingers is located at the amino terminus of the protein and a cluster of three fingers is located at the carboxyl terminus (see Remacle et al., EMBO J. (1999) 18(18):5073-84). Each cluster of zinc fingers in these proteins is able to bind to a unique target sequence and the spacing between the two target sequences can comprise many nucleotides.

The DNA-binding ZFP domains of the ZFP fusion proteins described herein direct the proteins to DNA target regions. In some embodiments, the DNA target region is at least 8 bps in length. For example, the target region may be 8 bps to 40 bps in length, such as 12, 13, 14, 15, 16, 17, 18, 19, 20, 21, 22, 23, 24, 25, 26, 27, 28, 29, 30, 31, 32, 33, 34, 35, or 36 bps in length.

In certain embodiments, the ZFP binds to a target site that is 1 to 100 (or any number therebetween) nucleotides on either side of the targeted base. In other embodiments, the ZFP binds to a target site that is 1 to 50 (or any number therebetween) nucleotides on either side of the targeted base.

In certain embodiments, a ZFP domain, or a ZFP fusion protein, of the present disclosure may be as described in the present Examples.

### C. Base Editor Inhibitors

In some embodiments, the base editor systems described herein may include an inhibitor of the editor to better regulate temporally and spatially the base editing activity of the systems. For example, where the cytidine deaminase is a TDD as described herein, the inhibitor may be a TDDI that inhibits said TDD. An example of a TDD/TDDI pair is the cytidine deaminase DddA and its inhibitor, DddI.

Thus, in some embodiments, the base editor systems include a TDDI component in addition to ZFP-TDD fusion proteins. The TDDI component may be brought in close proximity to the TDD complex through a DNA-binding domain covalently fused to it, or through dimerization with a DNA-binding domain not covalently bound to it.

In some embodiments, the present base editing system comprises a ZFP-inhibitor fusion protein comprising a ZFP domain and an inhibitor domain, wherein the ZFP domain binds to a sequence in the DNA target region close (e.g., within 50-100 nt) to the ZFP-cytidine deaminase fusion protein binding sites. When this ZFP-inhibitor fusion protein is introduced to the cell, the inhibitor domain will be brought within close proximity to the cytidine deaminase complex and bind to the complex, thereby inhibiting the base editing activity of the cytidine deaminase at that locus. The presence of the sequence bound by the ZFP domain of ZFP-inhibitor determines the inhibitory activity of the inhibitor.

In some embodiments, the binding of the inhibitor domain to the cytidine deaminase complex may be regulated by an agent (e.g., a small molecule or a peptide). For example, the inhibitor domain may be fused to a dimerization domain, and its dimerization partner may be fused to a ZFP domain that binds to a sequence in the DNA target region close (e.g., within 50-100 nt) to the ZFP-cytidine deaminase fusion protein binding sites. The dimerization domains of the inhibitor and the ZFP may dimerize in the presence of a dimerization-inducing agent (e.g., a small molecule or peptide). In the presence of the agent, the inhibitor domain will be brought within close proximity to the DNA target region through dimerization, leading to binding and inactivation of the cytidine deaminase complex. Once the agent is withdrawn, the inhibitor domain will no longer be sequestered near the DNA target region and will detach from the cytidine deaminase complex, allowing the base editing process to proceed. Examples of such agents and dimerizing domains are shown in Table 1 below:

TABLE 1
Dimerization Domains and Dimerization-Inducing Agents

| | | Dimerizing |
|---|---|---|
| Dimerization Partners | | Agent |
| FKBP | FKBP | FK1012 |
| FKBP | Calcineurin A (can) | FK506 |
| FKBP | CyP-Fas | FKCsA |
| FKBP | FRB (FKBP-rapamycin-binding) domain | Rapamycin |
| | of mTOR | |
| GyrB | GyrB | Coumermycin |
| GAI | GID1 (gibberellin insensitive dwarf 1) | Gibberellin |
| ABI | PYL | Abscisic acid |
| ABI | PYR^{Mandi} | Mandipropamid |
| SNAP-tag | HaloTag | HaXS |
| eDHFR | HaloTag | TMp-HTag |
| Bcl-xL | Fab (AZ1) | ABT-737 |

Conversely, the dimerization of the domains fused to the ZFP and the inhibitor domains may be inhibited, rather than promoted, by a dimerization-inhibiting agent (e.g., a small molecule or peptide) such that the presence of the agent will permit activity of the cytidine deaminase complex. If the agent is withdrawn, the inhibitor domain will be able to bind to the cytidine deaminase complex, inhibiting the base editing process.

### D. Uracil DNA Glycosylase Inhibitors

The term “uracil glycosylase inhibitor” or “UGI” as used herein, refers to a protein that can inhibit a uracil-DNA glycosylase base-excision repair enzyme. Upon detecting a G:U mismatch, the cell responds through base excision repair, initiated by excision of the mismatched uracil by uracil N-glycosylase (UNG). In some embodiments, a base editor system described herein further comprises one or more UGIs to protect the edited G:U intermediate from excision by UNG. In certain embodiments, a ZFP-cytidine deaminase (e.g., ZFP-TDD) fusion protein described herein may comprise one or more UGI domains, e.g., attached by a linker described herein. In some embodiments, the linker is an SGGS linker (SEQ ID NO: 95). The UGI domain(s) may be located at the N-terminus, the C-terminus, or any combination thereof, of the fusion protein (e.g., one UGI domain at the C-terminus, one UGI domain at the N-terminus, two UGI domains at the C-terminus, two UGI domains at the N-terminus, or any combination thereof). Additionally or alternatively, one or more UGI domains may be on a separate ZFP fusion protein (“ZFP-UGI”). In particular embodiments, the UGI domain comprises the amino acid sequence of SEQ ID NO: 99.

### E. Nickases

In some embodiments, a base editor system described herein further comprises a nickase to create a single-stranded DNA break in the vicinity of the edited DNA target region (e.g., within 10, 20, 30, 40, 50, 60, 70, 80, 90, or 100 nt from the edited base). The creation of the nick attracts DNA repair machinery such that the region downstream of the nick is excised and replaced, resulting in a fully edited double-stranded DNA target region. The nick may be, for example, 5′ or 3′ of the edited base on the same strand or the opposite strand. In certain embodiments, the nickase is part of a ZFP fusion protein.

In some embodiments, the nickase is comprised of first and second monomers (also referred to herein as nickase “half domains”) that dimerize to form an active nickase. One monomer may comprise a mutation rendering it catalytically inactive (resulting in nicking of only one DNA strand by the active monomer). In some embodiments, the monomers may be derived from FokI, e.g., FokI-ELD and FokI-KKR, wherein one of said monomers may have an inactivating mutation such as D450N. In particular embodiments, a pair of monomers forming a full active nickase may comprise a FokI-ELD-D450N monomer (e.g., having the amino acid sequence of SEQ ID NO: 109) and a FokI-KKR monomer (e.g., having the amino acid sequence of SEQ ID NO. 110).

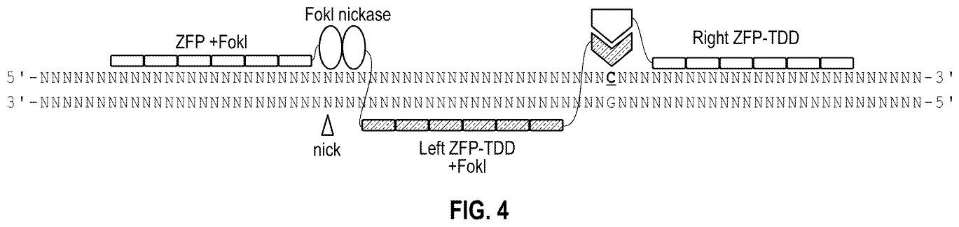

In some embodiments, the base editor system described herein has a trimeric architecture to include nickase function. For example, one half domain of a dimeric nickase may be fused to a ZFP-cytidine deaminase (e.g., a ZFP-TDD as described herein) and the other half domain may be fused to an independent ZFP, such that binding of both ZFP domains to their DNA target regions results in an active nickase capable of producing a single-strand break. See, e.g., FIG. 4.

In some embodiments, the base editor system described herein has a tetrameric architecture to include nickase function. In addition to the two ZFP-cytidine deaminase (e.g., ZFP-TDD as described herein) fusion proteins, such a system also comprises two ZFP-nickase proteins, wherein one half domain of a dimeric nickase is fused to a first ZFP domain and the other half domain fused to a second ZFP domain, such that binding of both ZFP domains to their DNA target regions results in an active nickase capable of producing a single-strand break.

In some embodiments, the nickase may be, for example, a ZFN nickase, a TALEN nickase, or a CRISPR/Cas nickase. In certain embodiments, the nickase is derived from a FokI DNA cleavage domain. In some embodiments, the FokI nickase comprises one or more mutations as compared to a parental FokI nickase, e.g., mutations to change the charge of the cleavage domain; mutations to residues that are predicted to be close to the DNA backbone based on molecular modeling and that show variation in FokI homologs; and/or mutations at other residues (see, e.g., U.S. Pat. No. 8,623,618 and Guo et al., J Mol Biol. (2010) 400(1):96-107). An exemplary pair of FokI half domains may be, e.g., SEQ ID NOs: 109 and 110.

In the ZFP fusion proteins described herein, the nickase domain(s) may be positioned on either side of the DNA-binding ZFP domain, including at the N- or C-terminal side of the fusion molecule (N- and/or C-terminal to the ZFP domain). In some embodiments, a ZFP-cytidine deaminase (e.g., ZFP-TDD as described herein) fusion protein described herein comprises a cytidine deaminase domain at the N- or C-terminus and a nickase domain at the opposite terminus.

### F. Peptide Linkers

In the fusion proteins described herein, the ZFP, cytidine deaminase (e.g., a TDD as described herein), inhibitor (e.g., a TDDI), nickase, and/or UGI domains may be positioned in any order relative to each other. In some embodiments, the domains may be associated with each other by direct peptidyl linkages, peptide linkers, or any combination thereof. In some embodiments, two or more of the domains may be associated with each other by dimerization (e.g., through a leucine zipper, a STAT protein N-terminal domain, or an FK506 binding protein).

In some embodiments, the ZFP, cytidine deaminase (e.g., a TDD as described herein), inhibitor (e.g., a TDDI), UGI, and/or nickase domains, and/or the zinc fingers within the ZFP domain, may be linked through a peptide linker, e.g., a noncleavable peptide linker of about 5 to 200 amino acids (e.g., 5, 6, 7, 8, 9, 10, 11, 12, 13, 14, 15, 16, 17, 18, 19, 20, 21, 22, 23, 24, 25, or 26 or more amino acids). Preferred linkers are typically flexible amino acid subsequences that are synthesized as a recombinant fusion protein. See, e.g., U.S. Pat. Nos. 6,479,626; 6,903,185; 7,153,949; 8,772,453; and 9,163,245; and PCT Patent Pub. WO 2011/139349. The proteins described herein may include any combination of suitable linkers.

In some embodiments, the peptide linker is three to 30 amino acid residues in length and is rich in G and/or S. Non-limiting examples of such linkers are SGGS linkers (SEQ ID NO: 95) as well as G4S-type linkers, i.e., linkers containing one or more (e.g., 2, 3, or 4) GGGGS (SEQ ID NO: 119) motifs, or variations of the motif (such as ones that have one, two, or three amino acid insertions, deletions, and/or substitutions from the motif).

In particular embodiments, a peptide linker used in a fusion protein described herein may be L26 (SEQ ID NO: 85), L21 (SEQ ID NO: 86), L18 (SEQ ID NO: 87), L13 (SEQ ID NO: 88), L11 (SEQ ID NO: 89), L9 (SEQ ID NO: 90), L7A (SEQ ID NO: 91), L6 (SEQ ID NO: 92), L4 (SEQ ID NO: 93), L0 (SEQ ID NO: 94), SGGS (SEQ ID NO: 95), or N6A (SEQ ID NO: 96), as shown in Table 4.

### II. Base Editor Systems

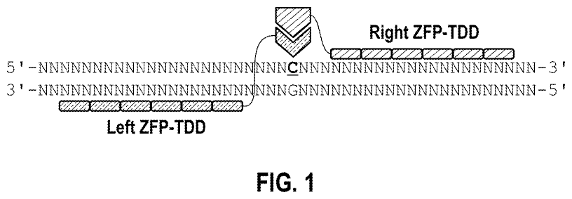

The present disclosure provides base editor systems comprising the ZFP fusion proteins described herein. The base editor systems can be used to edit a cytosine base to a uracil base in a DNA target region, wherein the uracil is replaced by a thymine base during DNA replication or repair. In certain embodiments, the editing results in the change of a targeted C:G base pair to a T:A base pair. FIG. 1 illustrates a base editing system of the present disclosure.

Base editor systems as described herein can be used to knock out a gene (e.g., by changing a regular codon into a stop codon and/or by mutating a splice acceptor site to introduce exon skipping and/or frameshift mutations); introduce mutations into a control element of a gene (e.g., a promoter or enhancer region) to increase or reduce expression; correct disease-causing mutations (e.g., point mutations); and/or induce mutations that result in therapeutic benefits. The target DNA may be in a chromosome or in an extrachromosomal sequence (e.g., mitochondrial DNA) in a cell. The base editing may be performed in vitro, ex vivo, or in vivo.

In some embodiments, a base editor system described herein performs one or more codon conversions, e.g., CAA to TAA; CAG to TAG; CGA to TGA; or TGG to TAG, TGA, or TAA; or any combination thereof, thereby introducing stop codon(s).

The base editor systems of the present disclosure may comprise, in addition to ZFP-cytidine deaminase (e.g., ZFP-TDD as described herein) fusion proteins, components such as inhibitor domains (e.g., a TDDI), UGIs, and nickases, or any combination thereof, as described herein that may help regulate or improve the editing activity of the system. In certain embodiments, the system may be packaged within a single viral vector (e.g., an AAV vector).

In some embodiments, a base editor system of the present disclosure comprises a pair of ZFP-cytidine deaminase (e.g., ZFP-TDD as described herein) fusion proteins each comprising a cytidine deaminase half domain that lacks cytidine deaminase activity on its own, wherein binding of the ZFPs to their respective nucleotide targets results in an active cytidine deaminase molecule capable of editing a targeted C base to T (e.g., by replacing C with U, which is replaced by T during DNA replication or repair).

For example, in some embodiments, the base editor system may comprise: a) a first fusion protein (ZFP-TDD left) comprising: i) a first ZFP domain that binds to nucleotides of a double-stranded DNA target region on one side of the base targeted for editing; and ii) a TDD N-half domain; and b) a second fusion protein (ZFP-TDD right) comprising: i) a second ZFP domain that binds to nucleotides of the double-stranded DNA target region on the other side of the base targeted for editing; and ii) a TDD C-half domain; wherein binding of the ZFP-TDD left and the ZFP-TDD right to their respective nucleotides results in an active TDD molecule capable of editing the DNA target region by changing the C base to T. The ZFP-TDDs and/or DNA target regions may be, e.g., as described herein.

In some embodiments, the base editor system described herein has a trimeric architecture to include nickase function. For example, the base editor system may comprise: first and second ZFP-TDD fusion proteins as described above, wherein binding of the ZFP-TDD left and the ZFP-TDD right to their respective nucleotides results in an active TDD molecule capable of editing the DNA target region by changing the C base to T. One half domain of a dimeric nickase may be fused to the first or second ZFP-TDD fusion protein, and the other half domain may be fused to an independent ZFP, such that binding of both ZFP domains to their DNA target regions results in an active nickase capable of producing a single-strand break. See, e.g., FIG. 4. The ZFP-TDDs, DNA target regions, and nickase domains may be, e.g., as described herein.

In some embodiments, the base editor system described herein has a tetrameric architecture to include nickase function. For example, the base editor system may comprise first and second ZFP-TDD fusion proteins as described above, wherein binding of the ZFP-TDD left and the ZFP-TDD right to their respective nucleotides results in an active TDD molecule capable of editing the DNA target region by changing the C base to T. In addition to the two ZFP-TDD fusion proteins, such a system also comprises two ZFP-nickase fusion proteins, wherein one half domain of a dimeric nickase is fused to a first ZFP domain and the other half domain fused to a second ZFP domain, such that binding of both ZFP domains to their DNA target regions results in an active nickase capable of producing a single-strand break. The ZFP-TDDs, DNA target regions, and nickase domains may be, e.g., as described herein.

In some embodiments, the base editor system may comprise: a) a first fusion protein (ZFP-TDDI) that binds to nucleotides within a first DNA target region, comprising: i) a zinc finger protein (ZFP) domain that binds to nucleotides within a first DNA target region; and ii) a TDDI domain; b) a second fusion protein (ZFP-TDD left) comprising: i) a ZFP domain that binds to nucleotides of a second DNA target region on one side of the base targeted for editing; and ii) a TDD N-half domain; and c) a third fusion protein (ZFP-TDD right) comprising: i) a ZFP domain that binds to nucleotides of the second DNA target region on the other side of the base targeted for editing; and ii) a TDD C-half domain; wherein binding of ZFP-TDD left and ZFP-TDD right to their respective nucleotides results in an active TDD molecule capable of editing the second DNA target region by changing the C base to T; and wherein binding of ZFP-TDDI to the first DNA target region prevents editing of the second DNA target region by the TDD. The ZFP-TDDs, ZFP-TDDI, and DNA target regions may be, e.g., as described herein.

In some embodiments, the base editor system may comprise: a) a first fusion protein comprising: i) a zinc finger protein (ZFP) domain that binds to nucleotides within a first DNA target region, and ii) a dimerization domain; b) a second fusion protein comprising: i) a TDDI domain; and ii) a dimerization domain that partners with the dimerization domain of a); c) a third fusion protein (ZFP-TDD left) comprising: i) a ZFP domain that binds to nucleotides of a second DNA target region on one side of the base targeted for editing, and ii) a TDD N-half domain; and d) a fourth fusion protein (ZFP-TDD right) comprising: i) a ZFP domain that binds to nucleotides of the second DNA target region on the other side of the base targeted for editing, and ii) a TDD C-half domain; wherein binding of ZFP-TDD left and ZFP-TDD right to their respective nucleotides results in an active TDD molecule capable of editing the second DNA target region by changing the C base to T; and wherein dimerization of the fusion proteins of a) and b) to form ZFP-TDDI and binding of the ZFP of a) to the first DNA target region prevents editing of the second DNA target region by the TDD. The ZFP-TDDs, ZFP-TDDI, and/or DNA target regions may be, e.g., as described herein.

In some embodiments, the dimerization domains of the fusion proteins of a) and b) partner to form ZFP-TDDI in the presence of a dimerization-inducing agent, resulting in inhibition of TDD activity.

In some embodiments, the dimerization domains of the fusion proteins of a) and b) are inhibited from partnering to form ZFP-TDDI in the presence of a dimerizing-inhibiting agent, permitting TDD activity.

In some embodiments, the ZFP-TDDI is specific for a sequence to be protected from TDD base editing activity. For example, the ZFP domain may bind to an allele to be preserved in its unedited form (e.g., where another allele, such as a mutated allele, is targeted for editing), or a known site of off-target editing. In some embodiments, the TDD base editing may convert a regular codon into a stop codon in the unprotected allele.

In some embodiments, expression of ZFP-TDDI (or components thereof) may be under the control of an inducible promoter. In certain embodiments, such a system may be used as a “kill switch,” wherein ZFP-TDDI protects an essential gene in a cell from being edited, and reducing or eliminating expression of ZFP-TDDI results in the death of the cell.

Where assembly of ZFP-TDDI is under the control of a dimerization-inducing or dimerization-inhibiting agent, base editing may be conditional upon the presence or absence of the agent. Such a conditional system may also be used for a “kill switch,” e.g., wherein ZFP-TDDI protects an essential gene in a cell from being edited in the presence of a dimerization-inducing agent or in the absence of a dimerization-inhibiting agent, and removing or administering the agent, respectively, results in the death of the cell.

In certain embodiments, a base editor system of the present disclosure may be a multiplex system comprising more than one ZFP-TDD left and ZFP-TDD right pair; such a system may be capable of editing more than one DNA target region at a time. In particular embodiments, to increase editing specificity, the multiplex system comprises ZFP-TDD pairs wherein the TDD N-half and C-half domains are split at a different position in the TDD sequence (e.g., a position described herein) for each pair. In certain embodiments, the DNA target regions edited by the ZFP-TDD pairs of the multiplex system may be in different genes. In certain embodiments, the DNA target regions may be in the same gene.

In any of the above embodiments, the TDD and TDDI may be any described herein. It is also contemplated that other cytidine deaminases and inhibitors may be used in place of the TDD and TDDI. In particular embodiments, a multiplex system described herein may comprise a first ZFP-cytidine deaminase pair and a second ZFP-cytidine deaminase pair, wherein the first and second pairs utilize different cytidine deaminases (e.g., selected from those described herein).

In some embodiments, the systems and methods described herein produce targeted editing of the DNA target region in at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95% or 100% of the cells. In some embodiments, the edited cells exhibit little to no off-target indels (e.g., less than 5%, 4%, 3%, 2%, 1%, 0.5%, 0.2%, or 0.10% off-target indels). In some embodiments, the edited cells exhibit little to no off-target base editing (e.g., less than 5%, 4%, 3%, 2%, 1%, 0.5%, 0.2%, or 0.1% off-target base editing); however, as base editing of off-target sites may not be prone to translocations or other genomic arrangements, higher percentages may also be contemplated.

The present disclosure also provides nucleic acid molecules encoding the ZFP fusion proteins described herein, which may be part of a viral or non-viral vector. Further, the present disclosure provides a cell or population of cells comprising a base editor system as described herein, as well as descendants of such cells, wherein the cells comprise one or more edited bases.

### III. Delivery of ZFP Fusion Proteins

A ZFP fusion protein of the present disclosure may be introduced to target cells as a protein, through a variety of methods (e.g., electroporation, fusion of the protein to a receptor ligand, lipid nanoparticles, cationic or anionic liposomes, or a nuclear localization signal (e.g., in combination with liposomes)). In other embodiments, the fusion protein is introduced to target cells through a nucleic acid molecule encoding it, for example, a DNA plasmid or mRNA. The nucleic acid molecule may be in a nucleic acid expression vector, which may include expression control sequences such as promoters, enhancers, transcription signal sequences, and transcription termination sequences that allow expression of the coding sequence for the ZFP fusion proteins. “Delivery of a system” as described herein may refer to either delivery of a system of fusion proteins as described herein or delivery of nucleic acid molecules encoding said system of fusion proteins or vectors or expression constructs comprising said nucleic acid molecules.

In some embodiments, the promoter on the vector for directing ZFP fusion protein expression is a constitutively active promoter or an inducible promoter. Suitable promoters include, without limitation, a Rous sarcoma virus (RSV) long terminal repeat (LTR) promoter (optionally with an RSV enhancer), a cytomegalovirus (CMV) promoter (optionally with a CMV enhancer), a CMV immediate early promoter, a simian virus 40 (SV40) promoter, a dihydrofolate reductase (DHFR) promoter, a β-actin promoter, a phosphoglycerate kinase (PGK) promoter, an EF1α promoter, a Moloney murine leukemia virus (MoMLV) LTR, a creatine kinase-based (CK6) promoter, a transthyretin promoter (TTR), a thymidine kinase (TK) promoter, a tetracycline responsive promoter (TRE), a hepatitis B Virus (HBV) promoter, a human α1-antitrypsin (hAAT) promoter, chimeric liver-specific promoters (LSPs), an E2 factor (E2F) promoter, the human telomerase reverse transcriptase (hTERT) promoter, a CMV enhancer/chicken β-actin/rabbit β-globin promoter (CAG promoter; Niwa et al., Gene (1991) 108(2):193-9), and an RU-486-responsive promoter. In addition, the promoter may include one or more self-regulating elements whereby the ZFP fusion protein can bind to and repress its own expression level to a preset threshold. See U.S. Pat. No. 9,624,498.

Any method of introducing the nucleotide sequence into a cell may be employed, including but not limited to, electroporation, calcium phosphate precipitation, microinjection, cationic or anionic liposomes, liposomes in combination with a nuclear localization signal, naturally occurring liposomes (e.g., exosomes), or viral transduction. In certain embodiments, the nucleotide sequence is in the form of mRNA and is delivered to a cell via electroporation.

For in vivo delivery of an expression vector, viral transduction may be used. A variety of viral vectors known in the art may be adapted by one of skill in the art for use in the present disclosure, for example, vaccinia vectors, adenoviral vectors, lentiviral vectors, poxyviral vectors, adeno-associated viral (AAV) vectors, retroviral vectors, and hybrid viral vectors. In some embodiments, the viral vector used herein is a recombinant AAV (rAAV) vector. Any suitable AAV serotype may be used. For example, the AAV may be AAV1, AAV2, AAV3, AAV3b, AAV4, AAV5, AAV6, AAV7, AAV8, AAV8.2, AAV9, AAV.PHP.B, AAV.PHP.eB, or AAVrh10, or of a novel serotype or a pseudotype such as AAV2/8, AAV2/5, AAV2/6, AAV2/9, or AAV2/6/9. In some embodiments, the expression vector is an AAV viral vector and is introduced to the target human cell by a recombinant AAV virion whose genome comprises the construct, including having the AAV Inverted Terminal Repeat (ITR) sequences on both ends to allow the production of the AAV virion in a production system such as an insect cell/baculovirus production system or a mammalian cell production system. The AAV may be engineered such that its capsid proteins have reduced immunogenicity or enhanced transduction ability in humans. Viral vectors described herein may be produced using methods known in the art. Any suitable permissive or packaging cell type may be employed to produce the viral particles. For example, mammalian (e.g., 293) or insect (e.g., sf9) cells may be used as the packaging cell line.

Any type of cell may be targeted for the base editing methods described herein. For example, the cells may be eukaryotic or prokaryotic. In some embodiments, the cells are mammalian (e.g., human) cells or plant cells. Human cells may include, for example, T cells, Natural Killer (NK) cells, NK T cells, alpha-beta T cells, gamma-delta T-cells, cytotoxic T lymphocytes (CTL), regulatory T cells, B cells, human embryonic stem cells, tumor-infiltrating lymphocytes (TIL) or a pluripotent stem cell from which lymphoid cells may be differentiated (e.g., an induced pluripotent stem cell (iPSC)). In some embodiments, the systems can be used to modify pluripotent stem cells prior to their differentiation into multiple cell types. For example, a lymphoid cell precursor may be modified prior to differentiation into lymphoid cell types such as regulatory T cells, effector T cells, natural killer cells, etc. The multiplex base editor systems of the present disclosure (comprising more than one ZFP-cytidine deaminase (e.g., ZFP-TDD) pair), in particular, can be used to prepare cells with multiple base edits at once, including pluripotent cells. In some embodiments, the multiplex systems may be used to prepare, e.g., allogeneic T cells. Where the systems comprise a ZFP-cytidine deaminase inhibitor (e.g., ZFP-TDDI) that can be induced to assemble in the presence or absence of a dimerization-regulating agent, as described herein, it is contemplated that the edited cells may be placed under the control of a “kill switch” activated upon administration of the agent.

For agricultural applications, any method for introduction of proteins or nucleic acid molecules to a plant cell is also contemplated, such as Agrobacterium tumefaciens-mediated T-DNA delivery.

### IV. Pharmaceutical Applications

The present disclosure provides methods of editing a cytosine to a thymine base in cellular DNA, comprising delivering a base editor system described herein to a cell (e.g., from a patient), resulting in the replacement of a targeted C base with a T base. The cell may be within a patient (in vivo treatment), or a method as described herein may be performed on a cell removed from a patient and then the edited cell delivered to the patient (ex vivo treatment). In some embodiments, the cells are further manipulated ex vivo prior to use as a treatment. The term “treating” encompasses alleviation of symptoms, prevention of onset of symptoms, slowing of disease progression, improvement of quality of life, and increased survival. In some embodiments, a patient treated by the methods described herein is a mammal, e.g., a human.

In some embodiments, the methods of the present disclosure are used to edit a gene or regulatory sequence associated with a disease. For example, in certain embodiments, the base editing may correct a point mutation in a DNA sequence to restore normal gene expression or activity. In certain embodiments, the base editing may introduce a stop codon into a deleterious gene (e.g., an oncogene). In certain embodiments, the base editing may introduce a mutation that results in a therapeutic benefit.

In some embodiments, the patient has cancer. In certain embodiments, the cell from the patient is further modified before or after base editing to provide resistance to a chemotherapeutic agent. The patient may then be treated with the chemotherapeutic agent, which in some embodiments may result in greater survival of edited over unedited cells.

In some embodiments, the patient has an autoimmune disorder.

In some embodiments, the patient has an autosomal dominant disease, such as autosomal dominant polycystic kidney disease.

In some embodiments, the patient has a mitochondrial disorder.

In some embodiments, the patient has sickle cell disease, hemophilia (e.g., hemophilia A, B, or C), cystic fibrosis, phenylketonuria, Tay-Sachs, prion disease, color blindness, a lysosomal storage disease (e.g., Fabry disease), Friedreich's ataxia, or prostate cancer.

In some embodiments, the methods of the present disclosure may target base editing to a particular allele of a gene, e.g., a wild-type or mutated allele. In certain embodiments, the allele may be associated with cancer. For example, the methods may target the V617F mutated allele of JAK2, which leads to constitutive tyrosine phosphorylation activity and plays a critical role in the expansion of myeloproliferative neoplasms. Knocking out expression of the allele with the V617F mutation, e.g., by introducing a stop codon, may facilitate successful treatment of JAK2 V617F disorders.

The present disclosure further provides a pharmaceutical composition comprising elements of a base editor system described herein, such as a ZFP-cytidine deaminase (e.g., ZFP-TDD as described herein) pair and optionally a nickase component (e.g., ZFP-nickase components for a trimeric or tetrameric system as described herein) and/or a cytidine deaminase inhibitor (e.g., TDDI) component (e.g., a ZFP-cytidine deaminase inhibitor component), or nucleotide sequences encoding said elements (e.g., in viral or non-viral vectors as described herein). The pharmaceutical composition may further comprise a pharmaceutically acceptable carrier such as water, saline (e.g., phosphate-buffered saline), dextrose, glycerol, sucrose, lactose, gelatin, dextran, albumin, or pectin. In addition, the composition may contain auxiliary substances, such as, wetting or emulsifying agents, pH-buffering agents, stabilizing agents, or other reagents that enhance the effectiveness of the pharmaceutical composition. The pharmaceutical composition may contain delivery vehicles such as liposomes, nanocapsules, microparticles, microspheres, lipid particles, and vesicles.

In some embodiments, the base editor systems described herein can be engineered to target to a genomic locus chosen from 2B4 (CD244), 4-1BB (CD137), A2aR, AAVS1, ACTB, AID, ALB, B2M, B7.1, B7.2, B7-H2, B7-H3, B7-H4, B7-H6, BAFFR, BCL11A, BLAME (SLAMF8), BTLA, butyrophilins, CIITA, CCR5, CD100 (SEMA4D), CD103, CD3zeta, CD4, CD5, CD7, CD11a, CD11b, CD11c, CD11d, CD150, IPO-3), CD160, CD160 (BY55), CD18, CD19, CD2, CD27, CD28, CD29, CD30, CD4, CD40, CD47, CD48, CD49a, CD49D, CD49f, CD52, CD69, CD7, CD83, CD84, CD8alpha, CD8beta, CD96 (Tactile), CDS, CEACAM1, CISH, CRTAM, CTLA4, CXCR4, DCK, DGK, DGKA, DGKB, DGKD, DGKE, DGKG, DGKI, DGKK, DGKQ, DGKZ, DHFR, DNAM1 (CD226), EP2/4 receptors, adenosine receptors including A2AR, FAS, FASLG, GADS, GITR, GM-CSF, gp49B, HHLA2, HLA-A, HLA-B, HLA-C, HLA-DPA1, HLA-DPB1, HIV-LTR (long terminal repeat), HLA-DQA1, HLA-DQB1, HLA-DRA, HLA-DRB1, HLA-I, HVEM, HVEM, IA4, ICAM-1, ICOS, ICOS, ICOS (CD278), IFN-alpha/beta/gamma, IL-1 beta, IL-12, IL-15, IL-18, IL-23, IL2R beta, IL2R gamma, IL2RA, IL-6, IL7R alpha, ILT-2, ILT-4, immunoglobulin heavy chain loci, immunoglobulin light chain loci, ITGA4, ITGA4, ITGA6, ITGAD, ITGAE, ITGAL, ITGAM, ITGAX, ITGB1, ITGB2, ITGB7, KIR family receptors, KLRG1, Lag-3, LAIR-1, LAT, LIGHT, LTBR, Ly9 (CD229), MNK1/2, NKG2C, NKG2D, NKp30, NKp44, NKp46, NKp80 (KLRF1), OX2R, OX40, PAG/Cbp, PD-1, PD-L1, PD-L2, PGE2 receptors, PIR-B, PPP1R12C, PRNP1, PSGL1, PTPN2, RANCE/RANKL, RFX5, ROSA26, SELPLG (CD162), SIRPalpha (CD47), SLAM (SLAMF1, SLAMF4 (CD244, 2B4), SLAMF5, SLAMF6 (NTB-A, Ly108), SLAMF7, SLP-76, SOCS1, SOCS3, Tetherin, TGFBR2, TIGIT, TIM-1, TIM-3, TIM-4, TMIGD2, TRA, TRAC, TRB, TRD, TRG, TNF, TNF-alpha, TNFR2, TRIM5, TUBAl, VISTA, VLA1, and VLA-6.

It is understood that the ZFP fusion proteins and base editor systems described herein may be used in a method of treatment described herein, may be for use in a treatment described herein, or may be used in the manufacture of a medicament for a treatment described herein.

### V. Agricultural Applications

The described systems and methods of editing a cytosine to a thymine base in cellular DNA may also be used in agricultural applications. For example, in certain embodiments, the base editing may correct one or more point mutations in a DNA sequence to restore normal gene expression or activity. In certain embodiments, the base editing may introduce a stop codon into one or more deleterious genes. In certain embodiments, the base editing may introduce one or more beneficial mutations. In particular embodiments, the systems and methods described herein are used to edit a crop plant.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cardiology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein.

Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It should also be noted that the term “or” is generally employed in its sense including “and/or” unless the content clearly dictates otherwise. As used herein the term “about” refers to a numerical range that is 10%, 5%, or 1% plus or minus from a stated numerical value within the context of the particular usage. Further, headings provided herein are for convenience only and do not interpret the scope or meaning of the claimed embodiments.

All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

### Example 1: Base Editing Activity of TDDs in Cells

Novel cytidine deaminases TDD20-TDD31 (Table 2) have been identified. These have been tested for base editing activity in K562 cells.

TABLE 2
TDD Information

| Ortholog No. | SEQ | Organism |
|---|---|---|
| TDD20 | 1 | Actinospica durhamensis |
| TDD21 | 2 | Brevilactibacter coleopterorum strain HDW11 |
| TDD22 | 3 | Candidatus promineofilum |
| TDD23 | 4 | Candidatus Melainabacteria |
| TDD24 | 5 | Falcatimonas |
| TDD25 | 6 | Hungatella xylanolytica |
| TDD26 | 7 | Frankiaceae |
| TDD27 | 8 | N/A |
| TDD28 | 9 | N/A |
| TDD29 | 10 | N/A |
| TDD30 | 11 | Treponema sp |
| TDD31 | 12 | N/A |

SEQ: SEQ ID NO:

To prepare ZFP-TDD fusion protein pairs, the TDD toxic domain was split into two halves each lacking cytidine deaminase activity (see Table 3).

TABLE 3
Sequences of TDD Toxic Domains and Splits

| TDD | Description | Sequence | SEQ |
|---|---|---|---|
| TDD20 | toxic domain | INGCGPSAVQQLLSANGEPGKTAGVLDLNGELTSLVSGKGE | 13 |
| | | LPNYAASGHVEGQAAMMMRAEKATSATLYIDNPNGICGYCR | |
| | | SQIATLLPEGATLEVVTPLGTVEPTARWSSSKVETGNERYP | |
| | | KGWVE | |
| | G2274-N | INGCGPSAVQQLLSANGEPGKTAGVLDLNGELTSLVSG | 25 |
| | G2274-C | KGELPNYAASGHVEGQAAMMMRAEKATSATLYIDNPNGICG | 26 |
| | | YCRSQIATLLPEGATLEVVTPLGTVEPTARWSSSKVETGNE | |
| | | RYPKGWVE | |
| | E2341-N | INGCGPSAVQQLLSANGEPGKTAGVLDLNGELTSLVSGKGE | 27 |
| | | LPNYAASGHVEGQAAMMMRAEKATSATLYIDNPNGICGYCR | |
| | | SQIATLLPEGATLEVVTPLGTVE | |
| | E2341-C | PTARWSSSKVETGNERYPKGWVE | 28 |
| | W2346-N | INGCGPSAVQQLLSANGEPGKTAGVLDLNGELTSLVSGKGE | 29 |
| | | LPNYAASGHVEGQAAMMMRAEKATSATLYIDNPNGICGYCR | |
| | | SQIATLLPEGATLEVVTPLGTVEPTARW | |
| | W2346-C | SSSKVETGNERYPKGWVE | 30 |
| TDD21 | toxic domain | MAAPGACTRTRLVTRSNSPCPPRAQASAGRGLAAPGTGAVA | 14 |
| | | SQSRGRRPVMDQTPPEARYVAAGHIEGKAALQVRAAASSGG | |
| | | TIHHNNPFGTCIYCQQQVPTLLPDGARLTAEPPAQARPRSA | |
| | | WWSPPGPVTMVGNVADPASHPVATAQ | |
| | G46-N | MAAPGACTRTRLVTRSNSPCPPRAQASAGRGLAAPGTGAVA | 31 |
| | | SQSRG | |
| | G46-C | RRPVMDQTPPEARYVAAGHIEGKAALQVRAAASSGGTIHHN | 32 |
| | | NPFGTCIYCQQQVPTLLPDGARLTAEPPAQARPRSAWWSPP | |
| | | GPVTMVGNVADPASHPVATAQ | |
| | S126-N | MAAPGACTRTRLVTRSNSPCPPRAQASAGRGLAAPGTGAVA | 33 |
| | | SQSRGRRPVMDQTPPEARYVAAGHIEGKAALQVRAAASSGG | |
| | | TIHHNNPFGTCIYCQQQVPTLLPDGARLTAEPPAQARPRSA | |
| | | WWS | |
| | S126-C | PPGPVTMVGNVADPASHPVATAQ | 34 |
| TDD22 | toxic domain | KASDGEEYLGHLPTRTRTRDKTHGLLVLDDEPPIPVVSGQN | 15 |
| | | GPAATIPRGSRGFDAITQTHVEGHVAALMVNHGWTRATLYI | |
| | | NNVPCPSCDRLLPRMLPPGARLNVIGPNGFQKLYVGGRW | |
| | G72-N | KASDGEEYLGHLPTRTRTRDKTHGLLVLDDEPPIPVVSG | 35 |
| | G72-C | QNGPAATIPRGSRGFDAITQTHVEGHVAALMVNHGWTRATL | 36 |
| | | YINNVPCPSCDRLLPRMLPPGARLNVIGPNGFQKLYVGGRW | |
| | G144-N | KASDGEEYLGHLPTRTRTRDKTHGLLVLDDEPPIPVVSGQN | 37 |
| | | GPAATIPRGSRGFDAITQTHVEGHVAALMVNHGWTRATLYI | |
| | | NNVPCPSCDRLLPRMLPPGARLNVIGPNG | |
| | G144-C | FQKLYVGGRW | 38 |
| TDD23 | toxic domain | NSVDRSYFERLPQRMRDSDKTAGILVADGQELDVLSGRKGP | 16 |
| | | AQSMPAQSRGFDLITKTHAEGHAIAHMIQHGIKEAKIYINN | |
| | | NEICSSCEKLIPRMLPPGSKLEVHMPSGAVRNEVGEPR | |
| | G313-N | NSVDRSYFERLPQRMRDSDKTAGILVADGQELDVLSG | 39 |
| | G313-C | RKGPAQSMPAQSRGFDLITKTHAEGHAIAHMIQHGIKEAKI | 40 |
| | | YINNNEICSSCEKLIPRMLPPGSKLEVHMPSGAVRNEVGEP | |
| | | R | |
| | G386-N | NSVDRSYFERLPQRMRDSDKTAGILVADGQELDVLSGRKGP | 41 |
| | | AQSMPAQSRGFDLITKTHAEGHAIAHMIQHGIKEAKIYINN | |
| | | NEICSSCEKLIPRMLPPGSKLEVHMPSG | |
| | G386-C | AVRNFVGEPR | 42 |
| TDD24 | toxic domain | DAAKAIKGGSSSINLPEYDGKTTHGVLVLDDGTQVPFSSGN | 17 |
| | | ANPNYKNYIPASHVEGKSAIYMRENGINNGTVFHNNTDGTC | |
| | | PYCDKMLPTLLEEGSTLTVVPPANANAPKPSWVDTVKTYIG | |
| | | NDKIPKKPK | |
| | G280-N | DAAKAIKGGSSSINLPEYDGKTTHGVLVLDDGTQVPFSSG | 43 |
| | G280-C | NANPNYKNYIPASHVEGKSAIYMRENGINNGTVFHNNTDGT | 44 |
| | | CPYCDKMLPTLLEEGSTLTVVPPANANAPKPSWVDTVKTYI | |
| | | GNDKIPKKPK | |
| | A349-N | DAAKAIKGGSSSINLPEYDGKTTHGVLVLDDGTQVPFSSGN | 45 |
| | | ANPNYKNYIPASHVEGKSAIYMRENGINNGTVFHNNTDGTC | |
| | | PYCDKMLPTLLEEGSTLTVVPPANANA | |
| | A349-C | PKPSWVDTVKTYIGNDKIPKKPK | 46 |
| | W354-N | DAAKAIKGGSSSINLPEYDGKTTHGVLVLDDGTQVPFSSGN | 47 |
| | | ANPNYKNYIPASHVEGKSAIYMRENGINNGTVFHNNTDGTC | |
| | | PYCDKMLPTLLEEGSTLTVVPPANANAPKPSW | |
| | W354-C | VDTVKTYIGNDKIPKKPK | 48 |
| TDD25 | toxic domain | SPDGHPIPRAAYNLPEYDGSTTHGVLITSDGSEISFTSGGG | 18 |
| | | DPRYTNYANNGHVEQKSSLYMRDNNISSGVVYHNNTEGTCG | |
| | | FCNTMTSTFLPEGATLTVVPPSNAVATNSKAVDVPKTYTGN | |
| | | SKDPKISSRYTGN | |
| | G88-N | SPDGHPIPRAAYNLPEYDGSTTHGVLITSDGSEISFTSG | 49 |
| | G88-C | GGDPRYTNYANNGHVEQKSSLYMRDNNISSGVVYHNNTEGT | 50 |
| | | CGFCNTMTSTFLPEGATLTVVPPSNAVATNSKAVDVPKTYT | |
| | | GNSKDPKISSRYTGN | |
| | A157-N | SPDGHPIPRAAYNLPEYDGSTTHGVLITSDGSEISFTSGGG | 51 |
| | | DPRYTNYANNGHVEQKSSLYMRDNNISSGVVYHNNTEGTCG | |
| | | FCNTMTSTFLPEGATLTVVPPSNAVA | |
| | A157-C | TNSKAVDVPKTYTGNSKDPKISSRYTGN | 52 |
| | A162-N | SPDGHPIPRAAYNLPEYDGSTTHGVLITSDGSEISFTSGGG | 53 |
| | | DPRYTNYANNGHVEQKSSLYMRDNNISSGVVYHNNTEGTCG | |
| | | FCNTMTSTFLPEGATLTVVPPSNAVATNSKA | |
| | A162-C | VDVPKTYTGNSKDPKISSRYTGN | 54 |
| TDD26 | toxic domain | TNADAQALLGSIRQAGKTAGVLNIDGDLMPLVSRKSSLPNY | 19 |
| | | AASGHVEGQAALIMRERGVSSAELLIDNPNGICSYCTSQVP | |
| | | TLLPEGAQLMVRPPLGTVPTQWWENGRTELGNAANPKPSPW | |
| | 3069-N | TNADAQALLGSIRQAGKTAGVLNIDGDLMPLVSRKSSLPNY | 55 |
| | | AASGHVEGQAALIMRERGVSSAELLIDNPNGICSYCTSQVP | |
| | | TLLPEGAQLMVRPPLGTVP | |
| | 3069-C | TQWWENGRTFLGNAANPKPSPW | 56 |
| | 3076-N | TNADAQALLGSIRQAGKTAGVLNIDGDLMPLVSRKSSLPNY | 57 |
| | | AASGHVEGQAALIMRERGVSSAELLIDNPNGICSYCTSQVP | |
| | | TLLPEGAQLMVRPPLGTVPTQWWENG | |
| | 3076-C | RTFLGNAANPKPSPW | 58 |
| TDD27 | toxic domain | ASSEYGEVPELPEFNGKKTEGVFRTADGKEIKFESGGSTEY | 20 |
| | | KNPSASHAEGKAAIYMRENGIKEGTVFHNNPNGTCNYCDKG | |
| | | LATLLPEGARLTVVPPIGAVAPNKYWVDVPKTYTGNGNLPS | |
| | | MK | |
| | G98-N | ASSEYGEVPELPEFNGKKTEGVERTADGKEIKFESG | 59 |
| | G98-C | GSTEYKNPSASHAEGKAAIYMRENGIKEGTVFHNNPNGTCN | 60 |
| | | YCDKGLATLLPEGARLTVVPPIGAVAPNKYWVDVPKTYTGN | |
| | | GNLPSMK | |
| | A165-N | ASSEYGEVPELPEFNGKKTEGVFRTADGKEIKFESGGSTEY | 61 |
| | | KNPSASHAEGKAAIYMRENGIKEGTVFHNNPNGTCNYCDKG | |
| | | LATLLPEGARLTVVPPIGAVA | |
| | A165-C | PNKYWVDVPKTYTGNGNLPSMK | 62 |
| | W170-N | ASSEYGEVPELPEFNGKKTEGVERTADGKEIKFESGGSTEY | 63 |
| | | KNPSASHAEGKAAIYMRENGIKEGTVFHNNPNGTCNYCDKG | |
| | | LATLLPEGARLTVVPPIGAVAPNKYW | |
| | W170-C | VDVPKTYTGNGNLPSMK | 64 |
| TDD28 | toxic domain | KLDVRNFFEDESETESGSSSINLPEYDGKTTHGVLVLDDGT | 21 |
| | | QVPFSSGNANPNYKNYIPASHVEGKSAIYMRENGINNGTVE | |
| | | HNNTDGTCPYCDKMLPTLLDEGSTLTVVPPTNASAPKPSWV | |
| | | DTVKTYIGNDKIPKKPK | |
| | G65-N | KLDVRNFFEDESETESGSSSINLPEYDGKTTHGVLVLDDGT | 65 |
| | | QVPFSSG | |
| | G65-C | NANPNYKNYIPASHVEGKSAIYMRENGINNGTVFHNNTDGT | 66 |
| | | CPYCDKMLPTLLDEGSTLTVVPPTNASAPKPSWVDTVKTYI | |
| | | GNDKIPKKPK | |
| | A181-N | KLDVRNFFEDESETESGSSSINLPEYDGKTTHGVLVLDDGT | 67 |
| | | QVPFSSGNANPNYKNYIPASHVEGKSAIYMRENGINNGTVE | |
| | | HNNTDGTCPYCDKMLPTLLDEGSTLTVVPPTNASA | |
| | A181-C | PKPSWVDTVKTYIGNDKIPKKPK | 68 |
| | W186-N | KLDVRNFFEDESETESGSSSINLPEYDGKTTHGVLVLDDGT | 69 |
| | | QVPFSSGNANPNYKNYIPASHVEGKSAIYMRENGINNGTVE | |
| | | HNNTDGTCPYCDKMLPTLLDEGSTLTVVPPTNASAPKPSW | |
| | W186-C | VDTVKTYIGNDKIPKKPK | 70 |
| TDD29 | toxic domain | YANIDKDKIDIPDFDGKITWGILVLEDGTCITESSGNANPM | 22 |
| | | FNHYIPASHAEGKAAIYMRQKGIKHGVIFHNNTDGTCPYCN | |
| | | TMLPTLLEENSTLIVVPPINAVAKKRGWIDKIKIYTGNNKI | |
| | | PKTN | |
| | G745-N | YANIDKDKIDIPDFDGKITWGILVLEDGTCITFSSG | 71 |
| | G745-C | NANPMENHYIPASHAEGKAAIYMRQKGIKHGVIFHNNTDGT | 72 |
| | | CPYCNTMLPTLLEENSTLIVVPPINAVAKKRGWIDKIKIYT | |
| | | GNNKIPKTN | |
| | W819-N | YANIDKDKIDIPDFDGKITWGILVLEDGTCITFSSGNANPM | 73 |
| | | FNHYIPASHAEGKAAIYMRQKGIKHGVIFHNNTDGTCPYCN | |
| | | TML PTLLEENSTLIVVPPINAVAKKRGW | |
| | W819-C | IDKIKIYTGNNKIPKTN | 74 |
| TDD30 | toxic domain | KKKNANKQKIELPPYDGKTTYGVLILDDGKQYSENSGKPAP | 23 |
| | | IYRNYIPASHVEGKAAIYMRENKIQSGTVYHNNTDGTCPYC | |
| | | DKMLPTLLEKDSTLKVVPPQNATSSKKGWITNEKIYIGNDK | |
| | | IPKTAR | |
| | G195-N | KKKNANKQKIELPPYDGKTTYGVLILDDGKQYSENSG | 75 |
| | G195-C | KPAPIYRNYIPASHVEGKAAIYMRENKIQSGTVYHNNTDGT | 76 |
| | | CPYCDKMLPTLLEKDSTLKVVPPQNATSSKKGWITNEKIYI | |
| | | GNDKIPKTAR | |
| | S264-N | KKKNANKQKIELPPYDGKTTYGVLILDDGKQYSENSGKPAP | 77 |
| | | IYRNYIPASHVEGKAAIYMRENKIQSGTVYHNNTDGTCPYC | |
| | | DKMLPTLLEKDSTLKVVPPQNATS | |
| | S264-C | SKKGWITNEKIYIGNDKIPKTAR | 78 |
| | W269-N | KKKNANKQKIELPPYDGKTTYGVLILDDGKQYSENSGKPAP | 79 |
| | | IYRNYIPASHVEGKAAIYMRENKIQSGTVYHNNTDGTCPYC | |
| | | DKMLPTLLEKDSTLKVVPPQNATSSKKGW | |
| | W269-C | ITNEKIYIGNDKIPKTAR | 80 |
| TDD31 | toxic domain | DMEAGLGESGRAHIALPEYDGGTTHGVLVLDDGTQIPLSSG | 24 |
| | | KGDPPGTNYAASGHVEGKAALIMRENGITSATLYINNPNGT | |
| | | CGYCDSQLPTLLPEGATLTVVPPANAVAPKSRWIDYPKTYI | |
| | | GNSNDPKPWPRYKGN | |
| | G41-N | DMEAGLGESGRAHIALPEYDGGTTHGVLVLDDGTQIPLSSG | 81 |
| | G41-C | KGDPPGTNYAASGHVEGKAALIMRENGITSATLYINNPNGT | 82 |
| | | CGYCDSQLPTLLPEGATLTVVPPANAVAPKSRWIDYPKTYI | |
| | | GNSNDPKPWPRYKGN | |
| | W115-N | DMEAGLGESGRAHIALPEYDGGTTHGVLVLDDGTQIPLSSG | 83 |
| | | KGDPPGTNYAASGHVEGKAALIMRENGITSATLYINNPNGT | |
| | | CGYCDSQLPTLLPEGATLTVVPPANAVAPKSRW | |
| | W115-C | IDYPKTYIGNSNDPKPWPRYKGN | 84 |

SEQ: SEQ ID NO:

### ZFP-TDD Design

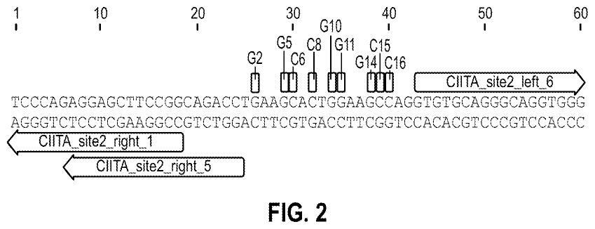

To assess editing at the human CIITA site 2 locus, left and right ZFP pairs were designed to target the TDD halves to a site at the locus (“site 2” as shown in FIG. 2), such that the halves dimerize at the target site and restore the catalytic activity of the TDD. FIG. 2 shows a left ZFP and two right ZFPs that can be used to form an exemplary left-right pair (e.g., CIITA_site_2_left 6+CIITA_site2_right 1 or CIITA_site_2_left 6+CIITA_site2_right 5). The present experiment used the pair CIITA_site_2_left 6+CIITA_site2 right 5.

The N-terminal half of each split TDD pair was fused to the C-terminus of the right ZFP and the C-terminal half was fused to the C-terminus of the left ZFP using the L26 linker (SEQ ID NO: 85). A UGI (uracil DNA glycosylase inhibitor) domain was fused to the C-terminus of each N-terminal and C-terminal half with an SGGLS linker (SEQ ID NO: 95). All ZFP-TDD fusion constructs further contained a 3×FLAG tag (e.g., SEQ ID NO: 97 or 98, or said sequence without the start codon) as well as an SV40 nuclear localization signal fused to the N-terminus of the ZFP.

The sequences for exemplary components, such as those included in the above-described ZFP-TDDs, are shown in Table 4 below.

TABLE 4
Sequences of ZFP-TDD Components

| SEQ | Description | Sequence |
|---|---|---|
| 100 | CIITA site 2 | RPFQCRICMRNFSRSAHLSRHIRTHTGEKPFACDICGRKFATSGH |
| | Left ZFP #6 | LSRHTKIHTHPRAPIPKPFQCRICMRNFSDSSHRTRHIRTHTGEK |
| | | PFACDICGRKFAAKWNLDAHTKIHTGSQKPFQCRICMRNFSRPYT |
| | | LRLHIRTHTGEKPFACDICGRKFALRHHLTRHTKIH |
| 101 | CIITA site 2 | RPFQCRICMRNFSRSAHLSRHIRTHTGEKPFACDICGRKFATSGH |
| | Left ZFP #6 | LSRHTKIHTHPRAPIPKPFQCRICMRNFSDSSHRTRHIRTHTGEK |
| | (variant) | PFACDICGRKFAAKWNLDAHTKIHTGSQKPFQCRICMRNFSRPYT |
| | | LRLHIRTHTGEKPFACDICGRKFALRHHLTRHTKIH |
| 102 | CIITA site 2 | ERPFQCRICMRNFSQSGHLARHIRTHTGEKPFACDICGRKFARKW |
| | Right ZFP #1 | TLQGHTKIHTGSQKPFQCRICMRNESIRSTLRDHIRTHTGEKPFA |
| | | CDICGRKFAHRSSLRRHTKIHTGSQKPFQCRICMRNFSQSGNLAR |
| | | HIRTHTGEKPFACDICGRKFARNVDLIHHTKIH |
| 103 | CIITA site 2 | ERPFQCRICMRNFSIRSTLRDHIRTHTGEKPFACDICGRKFAHRS |
| | Right ZFP #5 | SLRRHTKIHTGSQKPFQCRICMRNFSQSGNLARHIRTHTGEKPFA |
| | | CDICGRKFARNVDLIHHTKIHTGSQKPFQCRICMRNESRSDVLSE |
| | | HIRTHTGEKPFACDICGRKFATSGHLSRHTKIH |
| 85 | L26 linker | LRGSQLVKSKSEAAARGGGGSGGGGS |
| 86 | L21 linker | LRGSQLVKSKSEAAARGGGGS |
| 87 | L18 linker | LRGSQLVKSKSEAAARGS |
| 88 | L13 linker | LRGSQLVKSKSGS |
| 89 | L11 linker | LRGSQLVKSGS |
| 90 | L9 linker | LRGSQLVGS |
| 91 | L7A linker | LRGSQLVKSKSEAAAR |
| 92 | L6 linker | LRGSGS |
| 93 | L4 linker | LRGS |
| 94 | L0 linker | LRGSQLVKS |
| 95 | SGGS linker | SGGS |
| 96 | N6A linker | SGAQGSTLDE |
| 97 | 3xFlag + NLS | MDYKDHDGDYKDHDIDYKDDDDKMAPKKKRKVGIHGVPAAMA |
| 98 | 3xFlag + NLS | MDYKDHDGDYKDHDIDYKDDDDKMAPKKKRKVGIHGVPAAMGQLV |
| | (variant) | KS |
| 99 | UGI | TNLSDIIEKETGKQLVIQESILMLPEEVEEVIGNKPESDILVHTA |
| | | YDESTDENVMLLTSDAPEYKPWALVIQDSNGENKIKML |

SEQ: SEQ ID NO.

### TDD Base Editing Activity at the CIITA Locus

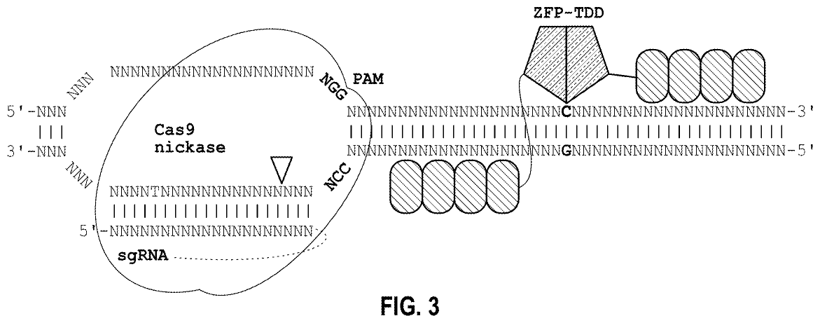

The base editing frequency of TDD20-TDD31 was tested at nucleotides G2, G5, C6, C8, G10, G11, G14, C15 and C16 of target sequence CIITA site 2 (FIG. 2). In some cases, the base editing system included a ZFP-FokI nickase to improve base editing activity. The efficiency of base editors can be increased by nicking the unmodified DNA strand with a nickase. The unmodified DNA strand then is recognized as newly synthesized by the cell, and the natural DNA repair machinery repairs the nicked DNA strand using the modified strand as a template. The unmodified strand can be nicked using a FokI-derived ZFN or TALEN or a CRISPR/Cas-derived nickase. However, all three approaches require the delivery of two additional constructs (two peptides for ZFN or TALEN nickases; one peptide and one sgRNA for CRISPR/Cas nickases; FIG. 3).

A trimeric ZFP-TDD base editor architecture may be used to overcome this limitation, facilitating delivery and also making it more likely that the base editing and DNA nicking will happen simultaneously, increasing editing efficiency. With such a trimeric architecture, one half domain of a dimeric nickase (e.g., FokI, wherein the halves may be, for example, SEQ ID NOs: 109 and 110) may be fused to the N-terminus of the left or right ZFP-TDD and the corresponding other half domain of the nickase may be targeted to the site of interest through an independent ZFP-nickase peptide (FIG. 4). The trimeric ZFP-TDD-nickase system was tested in K562 cells using the FokI nickase, according to the protocols described above.

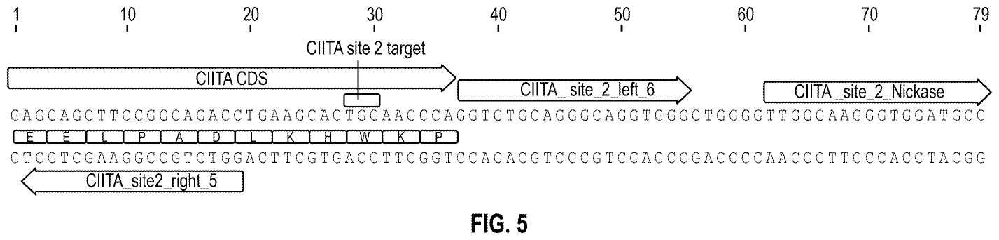

The ZFP design for the trimeric architecture at the CIITA locus is shown in FIG. 5. The three fusion proteins were constructed as follows:
1. CIITA_site_2_right_5 ZFP—TDD half domain #1;
2. Nickase half domain #1—CIITA site 2 left 6 ZFP—TDD half domain #2; and
3. CIITA_site_2_Nickase ZFP—Nickase half domain #2.

The fusion proteins were constructed as follows:

### Right ZFP #5-TDD Construct:

[3xFlag + NLS]-[CIITA_site_2_right_5 ZFP]-[L26 linker]-[TDD half domain #1]-
[SGGS]-[UGI]*,

specifically,

[SEQ ID NO: 97]-[SEQ ID NO: 103]-[SEQ ID NO: 85]-[TDD half domain #1]-[SEQ
ID NO: 95]-[SEQ ID NO: 99]-*

### Nickase-Left ZFP #6-TDD Construct:

[3xFLAG-NLS]-[Nickase half domain #1 (FokI-ELD_D450N)]-[N6A]-
[CIITA site_2_left_6 ZFP]-[L26 Linker]-[TDD half domain #2]-[SGGS]-[GI]-*;

specifically,

[SEQ ID NO: 112 without the start codon]-[SEQ ID NO: 96]-[SEQ ID NO: 101]-
[SEQ ID NO: 85]-[DD half domain #2]-[SEQ ID NO: 95]-[SEQ ID NO: 99]-*

### ZFP-Nickase Construct:

[3xFlag + NLS]-[IITA_site_2_Nickase ZFP]-[L0 Linker]-[Nickase half domain #2
(FokI-KKR)]-[Backbone]-*;

specifically, SEQ ID NO: 113.

Table 6 below includes certain sequences for these fusion proteins.

TABLE 6
CIITA Site 2 Trimeric Nickase Architecture Sequences

| SEQ | Description | Sequence |
|---|---|---|
| 109 | FokI-ELD D450N | ELEEKKSELRHKLKYVPHEYIELIEIARNSTQDRILEMKVME |
| | | FFMKVYGYRGKHLGGSRKPNGAIYTVGSPIDYGVIVDTKAYS |
| | | GGYNLPIGQADEMERYVEENQTRDKHLNPNEWWKVYPSSVTE |
| | | FKFLFVSGHFKGNYKAQLTRLNHITNCNGAVLSVEELLIGGE |
| | | MIKAGTLTLEEVRRKENNGEINF |
| 110 | FokI-KKR | ELEEKKSELRHKLKYVPHEYIELIEIARNSTQDRILEMKVME |
| | | FFMKVYGYRGKHLGGSRKPDGAIYTVGSPIDYGVIVDTKAYS |
| | | GGYNLPIGQADEMQRYVKENQTRNKHINPNEWWKVYPSSVTE |
| | | FKFLFVSGHFKGNYKAQLTRLNRKTNCNGAVLSVEELLIGGE |
| | | MIKAGTLTLEEVRRKENNGEINERS |
| 111 | CIITA site 2 | ERPFQCRICMRNESERGTLARHIRTHTGEKPFACDICGRKFA |
| | Nickase ZFP | TSANLSRHTKIHTGSQKPFQCRICMRNFSRSDVLSAHIRTHT |
| | | GEKPFACDICGRKFARNDHRINHTKIHTGSQKPFQCRICMRN |
| | | FSQSAHRKNHIRTHTGEKPFACDICGRKFARPYTLRLHTKIH |
| 112 | 3xFLAG-NLS-FokI- | MDYKDHDGDYKDHDIDYKDDDDKMAPKKKRKVGIHGVPAAMG |
| | ELD D450N | QLVKSELEEKKSELRHKLKYVPHEYIELIEIARNSTQDRILE |
| | | MKVMEFFMKVYGYRGKHLGGSRKPNGAIYTVGSPIDYGVIVD |
| | | TKAYSGGYNLPIGQADEMERYVEENQTRDKHLNPNEWWKVYP |
| | | SSVTEFKFLFVSGHFKGNYKAQLTRLNHITNCNGAVLSVEEL |
| | | LIGGEMIKAGTLTLEEVRRKENNGEINE |
| 113 | ZFP-FokI-KKR | MDYKDHDGDYKDHDIDYKDDDDKMAPKKKRKVGIHGVPAAMA |
| | | ERPFQCRICMRNESERGTLARHIRTHTGEKPFACDICGRKFA |
| | | TSANLSRHTKIHTGSQKPFQCRICMRNFSRSDVLSAHIRTHT |
| | | GEKPFACDICGRKFARNDHRINHTKIHTGSQKPFQCRICMRN |
| | | FSQSAHRKNHIRTHTGEKPFACDICGRKFARPYTLRLHTKIH |
| | | LRGSQLVKSELEEKKSELRHKLKYVPHEYIELIEIARNSTQD |
| | | RILEMKVMEFFMKVYGYRGKHLGGSRKPDGAIYTVGSPIDYG |
| | | VIVDTKAYSGGYNLPIGQADEMQRYVKENQTRNKHINPNEWW |
| | | KVYPSSVTEFKFLFVSGHFKGNYKAQLTRLNRKTNCNGAVLS |
| | | VEELLIGGEMIKAGTLTLEEVRRKENNGEINERS |

SEQ: SEQ ID NO.

To assay base editing in cells using constructs prepared as described above, K562 (ATCC, CCL243) cells were obtained from the ATCC and maintained in RPMI1640 with 10% FBS and 1× penicillin-streptomycin-glutamine (PSG) (Gibco, 10378-016) at 37° C. with 5% CO₂. 400 ng of pDNA encoding paired ZFP-TDDs was electroporated into K562 cells using the SF cell line 96-well Nucleofector kit (Lonza, V4SC-2960) following the manufacturer's instructions. In brief, cells were washed twice with 1×PBS (divalent cation-free) and resuspended at 2×10⁵ cells per 15 μL of supplemented SF cell line 96-well Nucleofector solution. For each transfection, 15 μL of the cell suspension was mixed with 5 μL of pDNA and transferred to the Lonza Nucleocuvette plate, then electroporated using the protocol for K562 cells (Nucleofector program 96-FF-120) on an Amaxa Nucleofector 96-well Shuttle System (Lonza). Electroporated cells were incubated at room temperature for 10 min and then transferred to 150 μL of prewarmed complete medium in a 96-well tissue culture plate. Cells were incubated for 72 h and then harvested for base editing quantification.

PCR primers for the CIITA site 2 locus were designed using Primer3 with the following optimal conditions: amplicon size of 200 nucleotides; a melting temperature of 60° C.; primer length of 20 nucleotides; and GC content of 50%. Sequences for the primers and amplicons are shown in Table 5 below.

TABLE 5
CIITA Site 2 Primer and Amplicon Sequences

| SEQ | Description | Sequence |
|---|---|---|
| 104 | CIITA site 2 | ACACGACGCTCTTCCGATCTNNNNCTGGGGCAGCTGATCACATGT |
| | forward primer | |
| 105 | CIITA site 2 | GACGTGTGCTCTTCCGATCTCTTCCATCCCCTCCCCAAG |
| | reverse primer | |
| 106 | CIITA site 2 | NNNNCTGGGGCAGCTGATCACATGTTTTCTCTGCAGCCTTCCCAGAG |
| | NGS amplicon | GAGCTTCCGGCAGACCTGAAGCACTGGAAGCCAGGTGTGCAGGGCAG |
| | | GTGGGCTGGGGTTGGGAAGGGTGGATGCCTTGGGGAGGGGATGGAAG |

SEQ: SEQ ID NO.

Adaptors were added for a second PCR reaction to add the Illumina library sequences (forward primer: ACACGACGCTCTTCCGATCT (SEQ ID NO: 107); reverse primer: GACGTGTGCTCTTCCGAT (SEQ ID NO: 108)). The CIITA site 2 locus was amplified in 25 μL using 100 ng of genomic DNA with AccuPrime HiFi (Invitrogen). Primers were used at a final concentration of 0.1 μM with the following thermocycling conditions: initial melt of 95° C. for 5 min; 35 cycles of 95° C. for 30 s, 55° C. for 30 s and 68° C. for 40 s; and a final extension at 68° C. for 10 min. PCR products were diluted 1:20 in water. 2 μL of diluted PCR product was used in a 20 μL PCR reaction to add the Illumina library sequences with Phusion High-Fidelity PCR MasterMix with HF Buffer (NEB). Primers were used at a final concentration of 0.5 μM with the following conditions: initial melt of 98° C. for 30 s; 12 cycles of 98° C. for 10 s, 60° C. for 30 s and 72° C. for 40 s; and a final extension at 72° C. for 10 min. A second PCR reaction was then performed to add sample specific sequence barcodes. PCR libraries were purified using the QIAquick PCR purification kit (Qiagen). Samples were quantified with the Qubit dsDNA HS Assay kit (Invitrogen) and diluted to 2 nM. The libraries were then run according to the manufacturer's instructions on either an Illumina MiSeq using a standard 300-cycle kit or an Illumina NextSeq 500 using a mid-output 300-cycle kit.

### Results

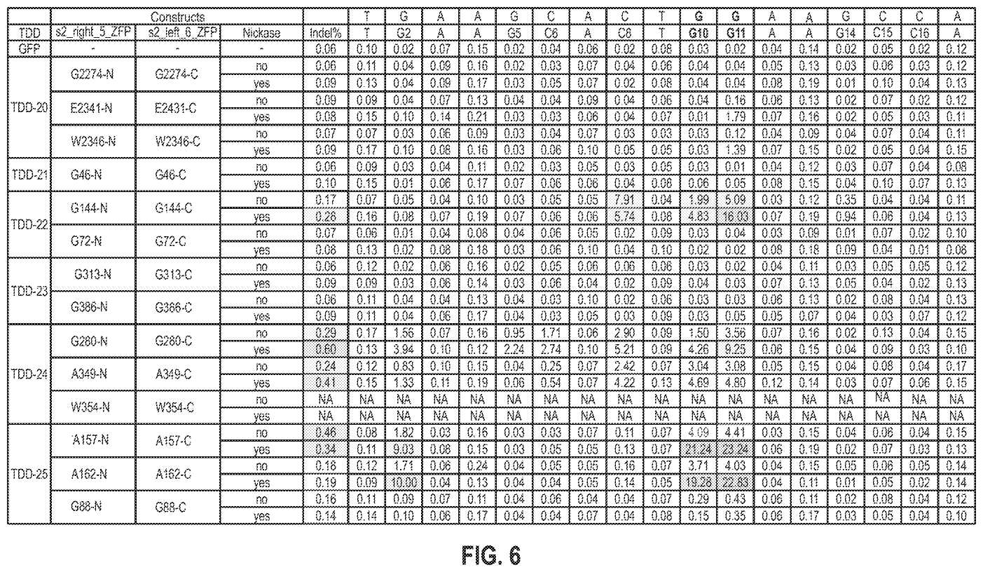

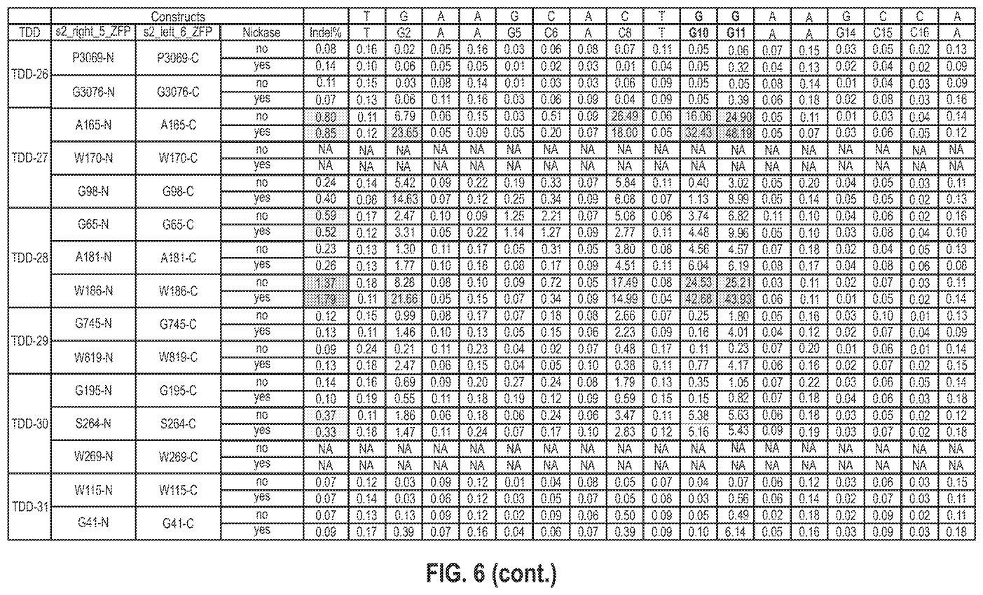

As shown in FIG. 6, at least 17 TDDs demonstrated detectable base editing activity (>0.25% base editing) at the CIITA locus, particularly at the CIITA site 2 target. In some cases, nicking appeared to improve editing activity.

### Example 2: Effect of Different Linkers on TDD Base Editing Activity at the CIITA Locus

To assess whether base editing activity is affected by different linkers between the deaminase and ZFP domains, the editing frequency at the CIITA locus of TDDs described herein may be assessed with different linkers, such as L26, L21, L18, L13, L11, L9, L6, and L4. It is contemplated that different linker lengths may be able to alter the base editing profile within the base editing window.

### Example 3: Targeting a TDD Inhibitor (TDDI) to a TDD

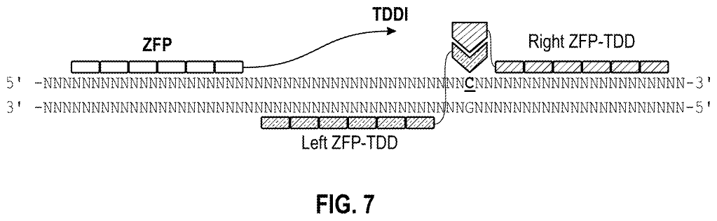

It is contemplated that TDDs described herein will be inactivated by TDD inhibitors (TDDIs). A ZFP or TALE linked TDDI can be targeted to a potential TDD-derived cytosine base editor site, preventing that site from being edited (FIG. 7). The TDDI may be linked to the ZFP using a dimerization domain potentiated by a small molecule, thus putting the editing activity under the control of the small molecule.

By designing the targeted TDDI construct to be allele specific, editing can be targeted selectively to certain alleles, e.g., to knock out a detrimental mutant by editing in a stop codon only if the mutation is present. For example, JAK2 V617F can be knocked out by editing in a stop codon only if the V617F mutation is present.

This TDDI approach may also be used to reduce editing at off-target sites, particularly where it cannot be eliminated by other means.

It is also contemplated that other cytidine deaminases and their inhibitors can be used in place of a TDD and TDDI.

What is claimed is:

1. A system for changing a cytosine to a thymine in the genome of a cell, comprising a first fusion protein and a second fusion protein, or first and second expression constructs for expressing the first and second fusion proteins, respectively, wherein
a) the first fusion protein comprises:
i) a first zinc finger protein (ZFP) domain that binds to a first sequence in a target genomic region in the cell, and
ii) a first portion of a cytidine deaminase polypeptide, wherein the cytidine deaminase is a toxin-derived deaminase (TDD) comprising an amino acid sequence at least 90% identical to any one of SEQ ID NOs: 13-24;
b) the second fusion protein comprises:
i) a second ZFP domain that binds to a second sequence in the target genomic region, and
ii) a second portion of the cytidine deaminase polypeptide;
c) the first and second portions lack cytidine deaminase activity on their own; and
d) binding of the first fusion protein and the second fusion protein to the target genomic region results in dimerization of the first and second portions, wherein the dimerized portions form an active cytidine deaminase capable of changing a cytosine to a thymine in the target genomic region,
optionally wherein the cell is a eukaryotic cell,
optionally wherein the eukaryotic cell is a mammalian cell or a plant cell,
further optionally wherein the mammalian cell is a human cell.

2. The system of claim 1, wherein the target genomic region is specific to a particular allele of a gene in the cell.

3. The system of claim 1, wherein the cytosine is between the proximal ends of the first sequence and the second sequence in the target genomic region, optionally wherein the proximal ends are no more than 100 bps apart.

4. The system of claim 1, comprising more than one pair of the first and second fusion proteins, wherein each pair of the fusion proteins binds to a different target genomic region.

5. The system of claim 4, wherein the first and second cytidine deaminase portions of one pair of fusion proteins are different from the first and second portions of another pair of fusion proteins.

6. The system of claim 1, further comprising a nickase that creates a single-stranded DNA break on the unedited or edited strand, wherein the DNA break is no more than about 500 bps, optionally no more than 200 bps, optionally about 10-50 bps, from the cytosine to be edited.

7. (canceled)

8. The system of claim 6, wherein the nickase is a ZFP-based nickase formed by dimerization of a first nickase domain and a second nickase domain fused respectively to two ZFP domains that bind to the target genomic region, wherein one of said nickase domains comprises an inactivating mutation.

9. The system of claim 7, wherein
one of the nickase domains is fused to the first or second fusion protein, and
the other nickase domain is fused to a third ZFP domain that binds to a third sequence in the target genomic region.

10. The system of claim 78, wherein the two nickase domains are fused respectively to
i) a third ZFP domain that binds a third sequence in the target genomic region and
ii) a fourth ZFP domain that binds a fourth sequence in the target genomic region.

11. The system of any one of claim 7, wherein the first and second nickase domains are derived from FokI.

12. The system of any one of claim 1, further comprising a third fusion protein or a third expression construct for expressing the third fusion protein in the cell, wherein
e) the third fusion protein comprises
i) a ZFP domain that binds to a third sequence in the target genomic region, and
ii) an inhibitory domain for the cytidine deaminase; and
f) binding of the third fusion protein to the target genomic region results in the inhibitory domain binding to, and thereby inhibition of the cytidine deaminase activity of, the dimerized cytidine deaminase portions.

13. The system of any one of claim 1, further comprising a third fusion protein or a third expression construct for expressing the third fusion protein in the cell, and a fourth fusion protein or a fourth expression construct for expressing the fourth fusion protein in the cell, wherein
e) the third fusion protein comprises
i) a ZFP domain that binds to a third sequence in the target genomic region, and
ii) a first dimerization domain; and
f) the fourth fusion protein comprises
i) an inhibitory domain for the cytidine deaminase, and
ii) a second dimerization domain capable of partnering with the first dimerization domain in the presence of a dimerization-inducing agent; and
g) binding of the third fusion protein to the target genomic region, and dimerization of the first and second dimerization domains, result in the inhibitory domain binding to, and thereby inhibition of the cytidine deaminase activity of, the dimerized cytidine deaminase portions.

14. The system of any one of claim 1, further comprising a third fusion protein or a third expression construct for expressing the third fusion protein in the cell, and a fourth fusion protein or a fourth expression construct for expressing the fourth fusion protein in the cell, wherein
e) the third fusion protein comprises
i) a ZFP domain that binds to a third sequence in the target genomic region, and
ii) a first dimerization domain; and
f) the fourth fusion protein comprises
i) an inhibitory domain for the cytidine deaminase, and
ii) a second dimerization domain capable of partnering with the first dimerization domain in the absence of a dimerization-inhibiting agent; and
g) binding of the third fusion protein to the target genomic region, and dimerization of the first and second dimerization domains, result in the inhibitory domain binding to, and thereby inhibition of the cytidine deaminase activity of, the dimerized cytidine deaminase portions.

15. (canceled)

16. (canceled)

17. (canceled)

18. (canceled)

19. (canceled)

20. The system of any one of claim 1, wherein the cytidine deaminase is a TDD that comprises an amino acid sequence at least 95% identical to the amino acid sequence of ay one of SEQ ID NOs: 13-24.

21. (canceled)

22. (canceled)

23. A fusion protein comprising i) a zinc finger protein (ZFP) domain that binds to a gene, and ii) a fragment of a cytidine deaminase polypeptide, wherein the cytidine deaminase is a toxin-derived deaminase (TDD) comprising an amino acid sequence at least 90% identical to any one of SEQ ID NOs: 13-24, optionally wherein the ZFP domain and the cytidine deaminase fragment are linked by a peptide linker, optionally wherein the gene is a eukaryotic gene, optionally wherein the eukaryotic gene is a human gene.

24. (canceled)

25. (canceled)

26. (canceled)

27. A pair of fusion proteins comprising
a) a first fusion protein that comprises i) a zinc finger protein (ZFP) domain that binds to a gene, and ii) a first dimerization domain, and
b) a second fusion protein that comprises i) a cytidine deaminase inhibitory domain, wherein the cytidine deaminase is a toxin-derived deaminase (TDD) comprising an amino acid sequence at least 90% identical to any one of SEQ ID NOs: 13-24, and ii) a second dimerization domain,
wherein the first and second dimerization domains can dimerize in the presence of a dimerization-inducing agent or in the absence of a dimerization-inhibiting agent,
optionally wherein the gene is a eukaryotic gene, optionally wherein the eukaryotic gene is a human gene.

28. (canceled)

29. (canceled)

30. One or more isolated nucleic acid molecules encoding the fusion protein(s) of any one of claim 15.

31. One or more expression constructs comprising the nucleic acid molecule(s) of claim 17.

32. One or more viral vectors comprising the expression construct(s) of claim 18, optionally wherein the viral vector is an adeno-associated viral vector, an adenoviral vector, or a lentiviral vector.

33. A cell comprising the system of claim 1.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)


SEQUENCE LISTING (119 sequences)

SEQ ID NO: 1 - protein, 2364 aa, Actinospica durhamensis
  REGION 1..2364: note = misc_feature - TDD20 (NCBI Accession No. WP_212528010.1)
MVGLVAGLALASPMPAVAAVAARPAASSGPAVTGVVMSSKSHTNTRTMTLNGKSVGVYKASGTQLPAATSGSATLQARGTATVSGNQPPAVTAAQLPKASIAGTPLWAQRTSTLSGPSSVTGAVASQSLAKQLGVTGVVYSVAGSGGSGSVRVGLDYAAFKDAYGANFGSRLELYTLPACALTTPQLAKCRVRTPVTGAVNDPAADTLSGVVKISGADTAASSGAAYTGGGTVSDGNYVIGTASAVSASSPSSGIVLAASSGAGEEGSATGNYAATKLSPAGSWTAGGSEGDFTYNYPITVPSSSTSLTPKVELDYDSGSVDGKTSMTNAQASMVGDGWTDPSENYITQTYVPCSDSPEGTASPTSTQDMCYDGEILSISLNGSSTTIVDDNGTFKLQNDNGAVISHVADSNKGQNTYNTDYWTVTERDGTEYYFGLNELPGYTSTGQTNSVDWEPVYAAHSGDPCWNATWADSVCNMAYEWHLDYVTDTHGDAMSYYYKQDTNYYGANNGASEKEYVRDSYLSEIDYGYTTASGAYGIVPDKVSFTTVNRCVASTCDAPSSSMSATTAASEYPDVPTDLICASGATCTSYSPSFFSTVMLTTITTSQYSLSASKQVDVDSYALAQDFPATGDNTSGTLWLESITHTGDDTSAGGSSSSISEPSVSFSGTDLPNRWDVETYPGLYRWRIADVTSELGSKVGVTYEIPDTCAASTLDAPTATPSSNTTSCYPVYWTPDGYSAAIEDWFIKYAVREVTVTDETGGAAEEVTQYSYANPAWHYDDEPAVQAKDRTYGEFRGYEQVNTVQGNGTSDAMDKSVTRYYQGMYGDYLSPTSTSTTTVPDTLGGVHNDYAALAGQPLETLTYFGDTSTVDKATVDSYWVSSATASQTFTGLPATTAQMTGPAEAYTEQLVTDSSTTSAWDYTETDDAYDATTTDADFGLKLYEYSHAWTSSGTADTDDTDTDYSHCTSYTYAPANTTLNIVGLSLATTEASVACSGFTESSIPSVPSTSTSLGAPASFTQDQVVSATLNFYDQNGSFVTTGIAPQTTTPTVGNLTETATATGYAPGTFTYQMASESTFDNYGRAEDTYDADGYKTITSYTVTDGITTAESVENALNQTTSETFDPARALVLTSTDINGVVTTKQYDALGRVTAVWGYSRATSTAANYLYSYTESKTGLSGSITEKLNDLADYTETATILDSLGRTRETQANTPAGGSLVTDTIYNSLGQVSATYNNWWDSTDLPSIALASSFDIDQIYNWDQYVYDGLGNKVEDESMTYDDQVYSTSYTVYSGDETTTFPPAGGTVKSTGTDPLGRTSSVSTYSTRPTVTIPSNVNTTPFYVSGGVALTTSYTYDGHGNQAVTTDPDGQKWTSVFDLLGRVTEKEDPTAGNSFTVYDADGNVLQTEDARSEYVSYTYDKLGRKTAEYASDDVAADQAKAGAEVAAWVYDDSNGLAAISAYAKGHVTTEYSYSGGATYTLQYEGFNVFGESLEEAYTFPSTVTGLAGTYYIGHSYTANLGLPASTIYPTTTGPLATETVGYSYTGALELPNGVNGTGQYSQSTAYDAWGRVSSETIGSTAATGEATLTNTYDPHTGALTEQLVTNATGNIDETTYAYDLYGNITKQLDERENTAADTETQCYQYNGLDQLSTAWTATDACATVPSQNSFSTVGDALGTASAYWDSWTYDNEGNRQTQDQHSLTSSAQDQITTDTISSTNNEISGTTTSVNSAQTASTTYGYDLAGNTDSRTTTADGTQTLSWNNEGQLYNISSTKNGTSAYVYDADGNLLTETDSATNSTTLYLPGEQITYNTGNGATSVNRYYALADGTAVRTGTGNNYDFELSDQHHTNDLYLDYTAQTPTWRQFDPFGNARGTAVTWVDNRTFLNDVTDTETGLTDIGARWYDAAIGRFESLDPVFEATDTLALGGYAYTDGNPITSSDPTGQMIEGESGYGTANDSQCDLSCATNLANNVTQKNQESIEAEHSHSGGLGTKVLALLCGVCAAVPEMYDQGAKIDPTNPAQGMINNADGETAIQQGIATDNPQELQNGFVQLGLTLAPGLEEAGALDDSSDLLMGACLGGESFTADTPVLTATGKQVAISTLKVGDKVEATNTSTGKTEPEKVSAVLVHHDTDLYDLTVRVGGKPSVIHTTSNHLYWVPGTGDKNHGGKWVTAAQLKPGTHLRTPDNATAVTVVAGATPAKPDGWMWDLTIPGDNDHDFYVVIGGTAVLVHNINGCGPSAVQQLLSANGEPGKTAGVLDLNGELTSLVSGKGELPNYAASGHVEGQAAMMMRAEKATSATLYIDNPNGICGYCRSQIATLLPEGATLEVVTPLGTVEPTARWSSSKVFTGNERYPKGWVE

SEQ ID NO: 2 - protein, 149 aa, Brevilactibacter coleopterorum
  REGION 1..149: note = misc_feature - TDD21 (NCBI Accession No. WP_166233360.1)
  source 1..149: note = strain HDW11
MAAPGACTRTRLVTRSNSPCPPRAQASAGRGLAAPGTGAVASQSRGRRPVMDQTPPEARYVAAGHIEGKAALQVRAAASSGGTIHHNNPFGTCIYCQQQVPTLLPDGARLTAEPPAQARPRSAWWSPPGPVTMVGNVADPASHPVATAQ

SEQ ID NO: 3 - protein, 154 aa, Candidatus promineofilum
  REGION 1..154: note = misc_feature - TDD22 (NCBI Accession No. MBK7217240.1)
MKHGRDSWYPIASDADLLVKTLLFASVADASFDKASDGEEYLGHLPTRTRTRDKTHGLLVLDDEPPIPVVSGQNGPAATIPRGSRGFDAITQTHVEGHVAALMVNHGWTRATLYINNVPCPSCDRLLPRMLPPGARLNVIGPNGFQKLYVGGRW

SEQ ID NO: 4 - protein, 396 aa, unidentified
  REGION 1..396: note = misc_feature - TDD23 (NCBI Accession No. RTL35118.1)
  source 1..396: note = Candidatus Melainabacteria
MPAEVTEKSQPAPPAQAESGMSQAAQSAWAPGSNYFRLGAVCNDTFNQSLSTQYDGLPQCAIAGIGEGLKKLSAIGEVGVGVAADVASVVVADLQIGYEAITYPSLAEGNSDRLAHKLRELQTKYEPLTQSVKQGYEGVQHDLNDIGAQSNNDSAAYARLVTDPAQSIVKAIDKFTGQTPAEQVKDIFDQGTQIAVAAALGKAAALASGSCRLEEIAAKFDALFSKMEETKQAFGTQLEHCIASGKKVQSSDYIRLSKAGDQDGLSRPADKISESCNSVDRSYFERLPQRMRDSDKTAGILVADGQELDVLSGRKGPAQSMPAQSRGFDLITKTHAEGHAIAHMIQHGIKEAKIYINNNEICSSCEKLIPRMLPPGSKLEVHMPSGAVRNFVGEPR

SEQ ID NO: 5 - protein, 372 aa, unidentified
  REGION 1..372: note = TDD24 (NCBI Accession No. WP_216577045.1), Falcatimonas sp.
RGSVRAVTDQTGTVTDRYSYSLYGERTHDTGDGDHIFGYCGRDGVITERDGLLYMRARYYSPELKRFISADSVIGNLSSTPSLNRYAYVNGNPVTQIDPFGLSAEPGTKKAKKNINWREAVHDALDIAGSITGYGDICDAVNAVLYLTEGDYINAGLSALSIIPGADAFTKTGKASKVLGNGIVESGLKHLDNVADGIKKLVKKADDAIDLEKHLPKKLLTEEAEAFAKKADKVADDVVEDAAKAIKGGSSSINLPEYDGKTTHGVLVLDDGTQVPFSSGNANPNYKNYIPASHVEGKSAIYMRENGINNGTVFHNNTDGTCPYCDKMLPTLLEEGSTLTVVPPANANAPKPSWVDTVKTYIGNDKIPKKPK

SEQ ID NO: 6 - protein, 185 aa, Hungatella xylanolytica
  REGION 1..185: note = misc_feature - TDD25 (NCBI Accession No. CP070896.1; bp 1412377-1412931)
FFYLKTAQRRAIIFLIEYENSPFVAYLSTPPEGSGQKNNGNTTTPDFYVSPDGHPIPRAAYNLPEYDGSTTHGVLITSDGSEISFTSGGGDPRYTNYANNGHVEQKSSLYMRDNNISSGVVYHNNTEGTCGFCNTMTSTFLPEGATLTVVPPSNAVATNSKAVDVPKTYTGNSKDPKISSRYTGN

SEQ ID NO: 7 - protein, 3091 aa, unidentified
  REGION 1..3091: note = TDD26 (NCBI Accession No. MBV9870847.1), Frankiaceae sp.
MARIQNGVNLHDGSTMDVPVTGVANVPTSGVTAVAISVTTQENNPQGGTLMVYPKGGTPSDPELPYPANGKFQSTTIFVQPDSVSGKITISNTNGDGYGVIVAVDVEGYFAANGSQNGFFPMIGERILFPGGGGVVDNTSIDATNPVTVKVAGVGGVPQNAASVAINIEATNTHFGAIYAYPSDANPAPSGVSELTMNTGVNADSLVQVSTTTSNSITIASTAAATIRLDVEGYTISGSNSPNAGAFVPLSDPQTVVPAQVVTSGNRIFQKVTNQDPGTAKDNIPSAASAVVGVLSAGSATAAGTARIWPSRQPAPGISQLEFGAAGAWSNLMIVPIGNEGQVNVRADLTGGTGSVAMTFVAIGYYTEAAPLPDTVSDPTPYLGNENYWTYARPTSVGPQSTAKVNVSNGNLVVSATDATPVPGHGHLDFNLKRTYNSLDNPGGTGVVDALEGLAGGFTKGTIGTGWTLSLGGFGTGVDSGFDTGGLTSLITPDWVAGTQQTVTLPTGQLILVDQDGTRHVFTADGAPLGAASNLLAKQGNIQTTGLGRTLGSLFKVTAAGNGSTLSQLLQITGNDKLDGSFCVYQPYNPPAGVHLSLFSVAYIQTVGTNDCVGGSNPVTVAYIAERPDGLRFVYSSGGQLLDEVDRAGDDLRFAYEGPLTAAVNGTTIANYGSLQHLPLVGDGSPINPSLKTVWEANVPGCTDGATGQFTGNSKCRSIGITYSERSEAGCTTTAPQTAVCVTDAAGRMTKYVLDGAPILSTPAHLVTVINPDDSTVNYTYGCNGGAVNQLCSVTDPNGGVATAISYLGTSAYSDSIDNGPLVSQVTDRRGIKTIFNYNVGHMAASAGAQFRGLSSTTEAVTQVALGDNCDLNVTASAGCRVSQYTAIDNRGRVGEIDRGTLSSSSYCSGQPAHPVDGAGICAQQVTTFGWDTRHLPIPSFAAKGSATAKATGGILPMGGGIKYSYVVTGVEGGGETPPSAVATTSSIATSSSGSVTLKWNAEVGASAYRIYGRTYNGTDMSTIKLIAEVPSTVNELNQPGSQFTWTDDGRTTPSNALPSTDTTQQVLCSAFDSNAVDNLLCRTTQLALNDNDTGQANLVFAADRATNYSYGLDGQPVLSDVVMPGSGGDLKTTYGYGYQYWDSAGDVVATGTHPTGSGNVSTPAFSSSDTLFTISDKTAMLPPRGNAAGGSYAPYETIYDSDDLSKVTDQQSQVTTYYPPNTVAGSGDPAGLSVCSTDGGTSGGKAGKPTANTGLMCEVDTPTPARASDAGGVSNCQAVDLAFAAVCTTYTYNGWGEKTSMTTPLAHTDDLGSANKFAAYHYVYYADTDTDVATATPSGGWLKAVVDPIAKKDDTVTDLSGLTQHFVVFGYDAAGNTVLTWDRYAVQRNTAADIAKGGTHDGIELSSYTPSESGGAPASGNQPGYSYQTYGSANYGASSSRDLFAKPWRYVVKSATAETSADPTVAPFEAPAPNYTTAPNETTVIVVDGNGNPTSITDPLGNATVNTYDAIDELKTSSTHAKTAGEKTTYVYDGDGNPITITSPNGQSGVAITTGNSPGFSTHIVYNALDLPIQTVTPRLANGDAGANLPAPFKGDKCSQGIGGYDALYDCQTVTVYDGFGEPVETIDGNNHVVYSFYDAAGRKIARSVSYGQPTSALNQADLNSQGTRSATPTGADYLNSAWVYDQDGNITKTCTPRDFDPNGGNAKAADNSTPGTSAPSTSAKLDTLTGACSNNVPLYGSTSTYGNADETTSVSKYRAPGSPATPDGTTVPSGTKALTTTSTYDADGNTVNVLDPAGTAGVAPNQASHVAMKYTYDSLDRKVCQSMPRLTSGLPAVPSDCTRTATQSAYTVTHYNDSGDVESVEAPKGDSASSAGGNLSRRSDTMYDLDHRVVATIAGATSADPFSTYLADKANQQDLRTGYYYDAGGNLIQQFDPRAYARANALKAPATRTDSNGPDPDYSVIIRYDGNHHPTKILSPRADSTLAHQSQPEGSQCPTSNAPTGSDVYKAFPTSPTTSVCETDVTYDRNGNRWTILLPTQVSGGQQPTVSYTYTYDDLLSSTATPSPTGTGSSTTTTYYDGDAQPVKTVDPDTKTTWTKYTGNELPYDVADADPNAIAPAKLLHHAITAYDANGATITSAGLVSNTGGWPNIGGSDGGGSWQVTTTAINSDGTTQTSTNTSRSMNGDTTTYDYDLNGNPTKIYSPKGSPPPASPTTVNTYTADNLLATSARSVTTSDSRTTSYTYDPAGRKEKQAIADGSLGAGGNLQFTYYPTDEPASQTGRDPSAAGNTTTTGTPDTFANGSGTVSFGYDEAGNQSTVNDSANYGGGSGSYGSSITNYYTLDSLLAETDEGPNADYLRYDGSGSVTMRTSQIGATRNQWIHFTNGPSGLPTAADTNQASGHWSFNYDSAGLLTDATYPDGEALHNNYYTGANNAATNYDSEMLKDTTLTSGVNTLQKFAYSYDGLDRIKTQNDQRAGGKLYTFSYDPQGRICQFDDGSATASTRNITWDADGNRTAFGNVGEENGCQGTGSNLTGSSTTTNYNLDDTIKTIGATSVAYDAAGRLTNDGVNNYCYDGFDQTELVGAASDTSCTTSVGTTYAYDGLSRQASSTKNSNTSKYLYDGTSSSLLTDSRSQGTAHGAINYLLSPTDAPLAIADDTNSGNITQYLTDDGAGNIGAVTDATTHLALCNPRYDPFGTLLPPAAGVPAPPASCPAYVGTLYYGANRRDANTGNYQDGARTYNPTLDAFTTADSYGPGDGSDDQSIGVDPLTANTYTYVNGDPINYDDPTGHYQAADTGAKPAAHGAVAPTYAQIVWELKYEEQQREEQIVHDLMSQVNSWSGCWSCIGSNLSQAGHDIASTATGVYNGAKNLASSCASGHWGTCGKGLGAIAIGVAAAFACPETAGATCALLAAGAAGAGGGALTCNGDSTANCAGKGALIGGLTFGIVKGGGAIRGVAGGAATNADAQALLGSIRQAGKTAGVLNIDGDLMPLVSRKSSLPNYAASGHVEGQAALIMRERGVSSAELLIDNPNGICSYCTSQVPTLLPEGAQLMVRPPLGTVPTQWWFNGRTFLGNAANPKPSPW

SEQ ID NO: 8 - protein, 187 aa, unidentified
  REGION 1..187: note = TDD27
MQGFVNAGELAIGDELLDSSGNILFVEKFDVELTEKPTKVYNFQVEDFHTYYVGENGVWVHNASSEYGEVPELPEFNGKKTEGVFRTADGKEIKFESGGSTEYKNPSASHAEGKAAIYMRENGIKEGTVFHNNPNGTCNYCDKGLATLLPEGARLTVVPPIGAVAPNKYWVDVPKTYTGNGNLPSMK

SEQ ID NO: 9 - protein, 204 aa, unidentified
  REGION 1..204: note = TDD28
MKKRLISFMLLITVISCVGFNTDYFKNLLTLISQAAKEVIYEETESYDEETSDSAVDRLEQELEKLDVRNFFEDESETESGSSSINLPEYDGKTTHGVLVLDDGTQVPFSSGNANPNYKNYIPASHVEGKSAIYMRENGINNGTVFHNNTDGTCPYCDKMLPTLLDEGSTLTVVPPTNASAPKPSWVDTVKTYIGNDKIPKKPK

SEQ ID NO: 10 - protein, 836 aa, unidentified
  REGION 1..836: note = TDD29
MRKKILKFILIQLMLVSFIFTNCGSMLKEMCTFAEQISSNEENTDKASIISCGDFNSDGKINVFDLMRSKQHVLNKEEQPSGLADINADGAFNILDSLILQDYILLRDTKIKDKVNVPPSDNIPDVSSDDSYRYVIFSEDKETPIQINSQLTTTNGKIATNNNFEGNRSINGDGKIIENANISMVYMDDKIVNTYFSNPEEYYEFVIENDDEANKIIDTPTISYDNILIEKSVTINSCMMAMNDIDIKSNTDNTGNTVIYSKYGDINIDCSNINLVGLIYAPFGTVKLSAQNVQIDGIIIAKNVIIDSEQKINLNPNNSIADFIGVDTEKMVIPYRDWCYIDKPDDAVLPEIVEEQIYEDPLSVDDAWLEDKYGSLIVDEYSAQMKKELVECLYICISERISNLSELIELFSFAAQGNAKDYVKLKTSLKNGKKDVSLTIGDYDISVEKEKSDKVIYGPMPYNPFVFRYYLTPNKTSDDSDNTDEPQEEPKKFKINDIGLFFELAAAGVSTAVVNVATTAGTAGTAVIGAGTAVIGAGTAAAATAINTVTTTVTKITTKAEQRIEQAVQNGKDSIVNIKNEVETSIKDTLSDIADVDLFGMQYVPLTNGFGYNIGSVRLNFIKEVGSWTSQTILNGEPINIDKIKTVVDLDTLNNINSISPQIYKDIASNIMNDKDLEKTYKIIDCINEKTDEFKLAYEQLKDIEQAYEYANIDKDKIDIPDFDGKITWGILVLEDGTCITFSSGNANPMFNHYIPASHAEGKAAIYMRQKGIKHGVIFHNNTDGTCPYCNTMLPTLLEENSTLIVVPPINAVAKKRGWIDKIKIYTGNNKIPKTN

SEQ ID NO: 11 - protein, 287 aa, unidentified
  REGION 1..287: note = TDD30 (NCBI Accession No. MBR1615955.1), Treponema sp.
MGNYESDKSRLTPYLFIVEVDGIQTMRFQKCEGLEAETEVFEYQEGGGTVHNFKGRSRFPNIVLEKGIVDSDDLWKWYNKTAEGNVERQSGSVVLCNLQGEEIKRWNFFRAFPCRWVGPALNAMDRRTFAVERIEIAHEWLEVDGGDEEEENLTSNNEKKKNANKQKIELPPYDGKTTYGVLILDDGKQYSFNSGKPAPIYRNYIPASHVEGKAAIYMRENKIQSGTVYHNNTDGTCPYCDKMLPTLLEKDSTLKVVPPQNATSSKKGWITNEKIYIGNDKIPKTAR

SEQ ID NO: 12 - protein, 138 aa, unidentified
  REGION 1..138: note = TDD31
DMEAGLGESGRAHIALPEYDGGTTHGVLVLDDGTQIPLSSGKGDPPGTNYAASGHVEGKAALIMRENGITSATLYINNPNGTCGYCDSQLPTLLPEGATLTVVPPANAVAPKSRWIDYPKTYIGNSNDPKPWPRYKGN

SEQ ID NO: 13 - protein, 128 aa, Actinospica durhamensis
  REGION 1..128: note = misc_feature - TDD20 toxic domain
INGCGPSAVQQLLSANGEPGKTAGVLDLNGELTSLVSGKGELPNYAASGHVEGQAAMMMRAEKATSATLYIDNPNGICGYCRSQIATLLPEGATLEVVTPLGTVEPTARWSSSKVFTGNERYPKGWVE

SEQ ID NO: 14 - protein, 149 aa, Brevilactibacter coleopterorum; identical to SEQ ID NO: 2
  REGION 1..149: note = misc_feature - TDD21 toxic domain
  source 1..149: note = strain HDW11

SEQ ID NO: 15 - protein, 121 aa, Candidatus promineofilum
  REGION 1..121: note = misc_feature - TDD22 toxic domain
KASDGEEYLGHLPTRTRTRDKTHGLLVLDDEPPIPVVSGQNGPAATIPRGSRGFDAITQTHVEGHVAALMVNHGWTRATLYINNVPCPSCDRLLPRMLPPGARLNVIGPNGFQKLYVGGRW

SEQ ID NO: 16 - protein, 120 aa, unidentified
  REGION 1..120: note = misc_feature - TDD23 toxic domain
  source 1..120: note = Candidatus Melainabacteria
NSVDRSYFERLPQRMRDSDKTAGILVADGQELDVLSGRKGPAQSMPAQSRGFDLITKTHAEGHAIAHMIQHGIKEAKIYINNNEICSSCEKLIPRMLPPGSKLEVHMPSGAVRNFVGEPR

SEQ ID NO: 17 - protein, 132 aa, unidentified
  REGION 1..132: note = TDD24 toxic domain
DAAKAIKGGSSSINLPEYDGKTTHGVLVLDDGTQVPFSSGNANPNYKNYIPASHVEGKSAIYMRENGINNGTVFHNNTDGTCPYCDKMLPTLLEEGSTLTVVPPANANAPKPSWVDTVKTYIGNDKIPKKPK

SEQ ID NO: 18 - protein, 136 aa, Hungatella xylanolytica
  REGION 1..136: note = misc_feature - TDD25 toxic domain
SPDGHPIPRAAYNLPEYDGSTTHGVLITSDGSEISFTSGGGDPRYTNYANNGHVEQKSSLYMRDNNISSGVVYHNNTEGTCGFCNTMTSTFLPEGATLTVVPPSNAVATNSKAVDVPKTYTGNSKDPKISSRYTGN

SEQ ID NO: 19 - protein, 123 aa, unidentified
  REGION 1..123: note = TDD26 toxic domain
TNADAQALLGSIRQAGKTAGVLNIDGDLMPLVSRKSSLPNYAASGHVEGQAALIMRERGVSSAELLIDNPNGICSYCTSQVPTLLPEGAQLMVRPPLGTVPTQWWFNGRTFLGNAANPKPSPW

SEQ ID NO: 20 - protein, 125 aa, unidentified
  REGION 1..125: note = TDD27 toxic domain
ASSEYGEVPELPEFNGKKTEGVFRTADGKEIKFESGGSTEYKNPSASHAEGKAAIYMRENGIKEGTVFHNNPNGTCNYCDKGLATLLPEGARLTVVPPIGAVAPNKYWVDVPKTYTGNGNLPSMK

SEQ ID NO: 21 - protein, 140 aa, unidentified
  REGION 1..140: note = TDD28 toxic domain
KLDVRNFFEDESETESGSSSINLPEYDGKTTHGVLVLDDGTQVPFSSGNANPNYKNYIPASHVEGKSAIYMRENGINNGTVFHNNTDGTCPYCDKMLPTLLDEGSTLTVVPPTNASAPKPSWVDTVKTYIGNDKIPKKPK

SEQ ID NO: 22 - protein, 127 aa, unidentified
  REGION 1..127: note = TDD29 toxic domain
YANIDKDKIDIPDFDGKITWGILVLEDGTCITFSSGNANPMFNHYIPASHAEGKAAIYMRQKGIKHGVIFHNNTDGTCPYCNTMLPTLLEENSTLIVVPPINAVAKKRGWIDKIKIYTGNNKIPKTN

SEQ ID NO: 23 - protein, 129 aa, unidentified
  REGION 1..129: note = TDD30 toxic domain
KKKNANKQKIELPPYDGKTTYGVLILDDGKQYSFNSGKPAPIYRNYIPASHVEGKAAIYMRENKIQSGTVYHNNTDGTCPYCDKMLPTLLEKDSTLKVVPPQNATSSKKGWITNEKIYIGNDKIPKTAR

SEQ ID NO: 24 - protein, 138 aa, unidentified; identical to SEQ ID NO: 12
  REGION 1..138: note = TDD31 toxic domain

SEQ ID NO: 25 - protein, 38 aa, Actinospica durhamensis
  REGION 1..38: note = misc_feature - TDD20 G2274-N
INGCGPSAVQQLLSANGEPGKTAGVLDLNGELTSLVSG

SEQ ID NO: 26 - protein, 90 aa, Actinospica durhamensis
  REGION 1..90: note = misc_feature - TDD20 G2274-C
KGELPNYAASGHVEGQAAMMMRAEKATSATLYIDNPNGICGYCRSQIATLLPEGATLEVVTPLGTVEPTARWSSSKVFTGNERYPKGWVE

SEQ ID NO: 27 - protein, 105 aa, Actinospica durhamensis
  REGION 1..105: note = misc_feature - TDD20 E2341-N
INGCGPSAVQQLLSANGEPGKTAGVLDLNGELTSLVSGKGELPNYAASGHVEGQAAMMMRAEKATSATLYIDNPNGICGYCRSQIATLLPEGATLEVVTPLGTVE

SEQ ID NO: 28 - protein, 23 aa, Actinospica durhamensis
  REGION 1..23: note = misc_feature - TDD20 E2341-C
PTARWSSSKVFTGNERYPKGWVE

SEQ ID NO: 29 - protein, 110 aa, Actinospica durhamensis
  REGION 1..110: note = misc_feature - TDD20 W2346-N
INGCGPSAVQQLLSANGEPGKTAGVLDLNGELTSLVSGKGELPNYAASGHVEGQAAMMMRAEKATSATLYIDNPNGICGYCRSQIATLLPEGATLEVVTPLGTVEPTARW

SEQ ID NO: 30 - protein, 18 aa, Actinospica durhamensis
  REGION 1..18: note = misc_feature - TDD20 W2346-C
SSSKVFTGNERYPKGWVE

SEQ ID NO: 31 - protein, 46 aa, Brevilactibacter coleopterorum
  REGION 1..46: note = misc_feature - TDD21 G46-N
  source 1..46: note = strain HDW11
MAAPGACTRTRLVTRSNSPCPPRAQASAGRGLAAPGTGAVASQSRG

SEQ ID NO: 32 - protein, 103 aa, Brevilactibacter coleopterorum
  REGION 1..103: note = misc_feature - TDD21 G46-C
  source 1..103: note = strain HDW11
RRPVMDQTPPEARYVAAGHIEGKAALQVRAAASSGGTIHHNNPFGTCIYCQQQVPTLLPDGARLTAEPPAQARPRSAWWSPPGPVTMVGNVADPASHPVATAQ

SEQ ID NO: 33 - protein, 126 aa, Brevilactibacter coleopterorum
  REGION 1..126: note = misc_feature - TDD21 S126-N
  source 1..126: note = strain HDW11
MAAPGACTRTRLVTRSNSPCPPRAQASAGRGLAAPGTGAVASQSRGRRPVMDQTPPEARYVAAGHIEGKAALQVRAAASSGGTIHHNNPFGTCIYCQQQVPTLLPDGARLTAEPPAQARPRSAWWS

SEQ ID NO: 34 - protein, 23 aa, Brevilactibacter coleopterorum
  REGION 1..23: note = misc_feature - TDD21 S126-C
  source 1..23: note = strain HDW11
PPGPVTMVGNVADPASHPVATAQ

SEQ ID NO: 35 - protein, 39 aa, Candidatus promineofilum
  REGION 1..39: note = misc_feature - TDD22 G72-N
KASDGEEYLGHLPTRTRTRDKTHGLLVLDDEPPIPVVSG

SEQ ID NO: 36 - protein, 82 aa, Candidatus promineofilum
  REGION 1..82: note = misc_feature - TDD22 G72-C
QNGPAATIPRGSRGFDAITQTHVEGHVAALMVNHGWTRATLYINNVPCPSCDRLLPRMLPPGARLNVIGPNGFQKLYVGGRW

SEQ ID NO: 37 - protein, 111 aa, Candidatus promineofilum
  REGION 1..111: note = misc_feature - TDD22 G144-N
KASDGEEYLGHLPTRTRTRDKTHGLLVLDDEPPIPVVSGQNGPAATIPRGSRGFDAITQTHVEGHVAALMVNHGWTRATLYINNVPCPSCDRLLPRMLPPGARLNVIGPNG

SEQ ID NO: 38 - protein, 10 aa, Candidatus promineofilum
  REGION 1..10: note = misc_feature - TDD22 G144-C
FQKLYVGGRW

SEQ ID NO: 39 - protein, 37 aa, unidentified
  REGION 1..37: note = misc_feature - TDD23 G313-N
  source 1..37: note = Candidatus Melainabacteria
NSVDRSYFERLPQRMRDSDKTAGILVADGQELDVLSG

SEQ ID NO: 40 - protein, 83 aa, unidentified
  REGION 1..83: note = misc_feature - TDD23 G313-C
  source 1..83: note = Candidatus Melainabacteria
RKGPAQSMPAQSRGFDLITKTHAEGHAIAHMIQHGIKEAKIYINNNEICSSCEKLIPRMLPPGSKLEVHMPSGAVRNFVGEPR

SEQ ID NO: 41 - protein, 110 aa, unidentified
  REGION 1..110: note = misc_feature - TDD23 G386-N
  source 1..110: note = Candidatus Melainabacteria
NSVDRSYFERLPQRMRDSDKTAGILVADGQELDVLSGRKGPAQSMPAQSRGFDLITKTHAEGHAIAHMIQHGIKEAKIYINNNEICSSCEKLIPRMLPPGSKLEVHMPSG

SEQ ID NO: 42 - protein, 10 aa, unidentified
  REGION 1..10: note = misc_feature - TDD23 G386-C
  source 1..10: note = Candidatus Melainabacteria
AVRNFVGEPR

SEQ ID NO: 43 - protein, 40 aa, unidentified
  REGION 1..40: note = TDD24 G280-N
DAAKAIKGGSSSINLPEYDGKTTHGVLVLDDGTQVPFSSG

SEQ ID NO: 44 - protein, 92 aa, unidentified
  REGION 1..92: note = TDD24 G280-C
NANPNYKNYIPASHVEGKSAIYMRENGINNGTVFHNNTDGTCPYCDKMLPTLLEEGSTLTVVPPANANAPKPSWVDTVKTYIGNDKIPKKPK

SEQ ID NO: 45 - protein, 109 aa, unidentified
  REGION 1..109: note = TDD24 A349-N
DAAKAIKGGSSSINLPEYDGKTTHGVLVLDDGTQVPFSSGNANPNYKNYIPASHVEGKSAIYMRENGINNGTVFHNNTDGTCPYCDKMLPTLLEEGSTLTVVPPANANA

SEQ ID NO: 46 - protein, 23 aa, unidentified
  REGION 1..23: note = TDD24 A349-C
PKPSWVDTVKTYIGNDKIPKKPK

SEQ ID NO: 47 - protein, 114 aa, unidentified
  REGION 1..114: note = TDD24 W354-N
DAAKAIKGGSSSINLPEYDGKTTHGVLVLDDGTQVPFSSGNANPNYKNYIPASHVEGKSAIYMRENGINNGTVFHNNTDGTCPYCDKMLPTLLEEGSTLTVVPPANANAPKPSW

SEQ ID NO: 48 - protein, 18 aa, unidentified
  REGION 1..18: note = TDD24 W354-C
VDTVKTYIGNDKIPKKPK

SEQ ID NO: 49 - protein, 39 aa, Hungatella xylanolytica
  REGION 1..39: note = misc_feature - TDD25 G88-N
SPDGHPIPRAAYNLPEYDGSTTHGVLITSDGSEISFTSG

SEQ ID NO: 50 - protein, 97 aa, Hungatella xylanolytica
  REGION 1..97: note = misc_feature - TDD25 G88-C
GGDPRYTNYANNGHVEQKSSLYMRDNNISSGVVYHNNTEGTCGFCNTMTSTFLPEGATLTVVPPSNAVATNSKAVDVPKTYTGNSKDPKISSRYTGN

SEQ ID NO: 51 - protein, 108 aa, Hungatella xylanolytica
  REGION 1..108: note = misc_feature - TDD25 A157-N
SPDGHPIPRAAYNLPEYDGSTTHGVLITSDGSEISFTSGGGDPRYTNYANNGHVEQKSSLYMRDNNISSGVVYHNNTEGTCGFCNTMTSTFLPEGATLTVVPPSNAVA

SEQ ID NO: 52 - protein, 28 aa, Hungatella xylanolytica
  REGION 1..28: note = misc_feature - TDD25 A157-C
TNSKAVDVPKTYTGNSKDPKISSRYTGN

SEQ ID NO: 53 - protein, 113 aa, Hungatella xylanolytica
  REGION 1..113: note = misc_feature - TDD25 A162-N
SPDGHPIPRAAYNLPEYDGSTTHGVLITSDGSEISFTSGGGDPRYTNYANNGHVEQKSSLYMRDNNISSGVVYHNNTEGTCGFCNTMTSTFLPEGATLTVVPPSNAVATNSKA

SEQ ID NO: 54 - protein, 23 aa, Hungatella xylanolytica
  REGION 1..23: note = misc_feature - TDD25 A162-C
VDVPKTYTGNSKDPKISSRYTGN

SEQ ID NO: 55 - protein, 101 aa, unidentified
  REGION 1..101: note = TDD26 3069-N
TNADAQALLGSIRQAGKTAGVLNIDGDLMPLVSRKSSLPNYAASGHVEGQAALIMRERGVSSAELLIDNPNGICSYCTSQVPTLLPEGAQLMVRPPLGTVP

SEQ ID NO: 56 - protein, 22 aa, unidentified
  REGION 1..22: note = TDD26 3069-C
TQWWFNGRTFLGNAANPKPSPW

SEQ ID NO: 57 - protein, 108 aa, unidentified
  REGION 1..108: note = TDD26 3076-N
TNADAQALLGSIRQAGKTAGVLNIDGDLMPLVSRKSSLPNYAASGHVEGQAALIMRERGVSSAELLIDNPNGICSYCTSQVPTLLPEGAQLMVRPPLGTVPTQWWFNG

SEQ ID NO: 58 - protein, 15 aa, unidentified
  REGION 1..15: note = TDD26 3076-C
RTFLGNAANPKPSPW

SEQ ID NO: 59 - protein, 36 aa, unidentified
  REGION 1..36: note = TDD27 G98-N
ASSEYGEVPELPEFNGKKTEGVFRTADGKEIKFESG

SEQ ID NO: 60 - protein, 89 aa, unidentified
  REGION 1..89: note = TDD27 G98-C
GSTEYKNPSASHAEGKAAIYMRENGIKEGTVFHNNPNGTCNYCDKGLATLLPEGARLTVVPPIGAVAPNKYWVDVPKTYTGNGNLPSMK

SEQ ID NO: 61 - protein, 103 aa, unidentified
  REGION 1..103: note = TDD27 A165-N
ASSEYGEVPELPEFNGKKTEGVFRTADGKEIKFESGGSTEYKNPSASHAEGKAAIYMRENGIKEGTVFHNNPNGTCNYCDKGLATLLPEGARLTVVPPIGAVA

SEQ ID NO: 62 - protein, 22 aa, unidentified
  REGION 1..22: note = TDD27 A165-C
PNKYWVDVPKTYTGNGNLPSMK

SEQ ID NO: 63 - protein, 108 aa, unidentified
  REGION 1..108: note = TDD27 W170-N
ASSEYGEVPELPEFNGKKTEGVFRTADGKEIKFESGGSTEYKNPSASHAEGKAAIYMRENGIKEGTVFHNNPNGTCNYCDKGLATLLPEGARLTVVPPIGAVAPNKYW

SEQ ID NO: 64 - protein, 17 aa, unidentified
  REGION 1..17: note = TDD27 W170-C
VDVPKTYTGNGNLPSMK

SEQ ID NO: 65 - protein, 48 aa, unidentified
  REGION 1..48: note = TDD28 G65-N
KLDVRNFFEDESETESGSSSINLPEYDGKTTHGVLVLDDGTQVPFSSG

SEQ ID NO: 66 - protein, 92 aa, unidentified
  REGION 1..92: note = TDD28 G65-C
NANPNYKNYIPASHVEGKSAIYMRENGINNGTVFHNNTDGTCPYCDKMLPTLLDEGSTLTVVPPTNASAPKPSWVDTVKTYIGNDKIPKKPK

SEQ ID NO: 67 - protein, 117 aa, unidentified
  REGION 1..117: note = TDD28 A181-N
KLDVRNFFEDESETESGSSSINLPEYDGKTTHGVLVLDDGTQVPFSSGNANPNYKNYIPASHVEGKSAIYMRENGINNGTVFHNNTDGTCPYCDKMLPTLLDEGSTLTVVPPTNASA

SEQ ID NO: 68 - protein, 23 aa, unidentified
  REGION 1..23: note = TDD28 A181-C
PKPSWVDTVKTYIGNDKIPKKPK

SEQ ID NO: 69 - protein, 122 aa, unidentified
  REGION 1..122: note = TDD28 W186-N
KLDVRNFFEDESETESGSSSINLPEYDGKTTHGVLVLDDGTQVPFSSGNANPNYKNYIPASHVEGKSAIYMRENGINNGTVFHNNTDGTCPYCDKMLPTLLDEGSTLTVVPPTNASAPKPSW

SEQ ID NO: 70 - protein, 18 aa, unidentified
  REGION 1..18: note = TDD28 W186-C
VDTVKTYIGNDKIPKKPK

SEQ ID NO: 71 - protein, 36 aa, unidentified
  REGION 1..36: note = TDD29 G745-N
YANIDKDKIDIPDFDGKITWGILVLEDGTCITFSSG

SEQ ID NO: 72 - protein, 91 aa, unidentified
  REGION 1..91: note = TDD29 G745-C
NANPMFNHYIPASHAEGKAAIYMRQKGIKHGVIFHNNTDGTCPYCNTMLPTLLEENSTLIVVPPINAVAKKRGWIDKIKIYTGNNKIPKTN

SEQ ID NO: 73 - protein, 110 aa, unidentified
  REGION 1..110: note = TDD29 W819-N
YANIDKDKIDIPDFDGKITWGILVLEDGTCITFSSGNANPMFNHYIPASHAEGKAAIYMRQKGIKHGVIFHNNTDGTCPYCNTMLPTLLEENSTLIVVPPINAVAKKRGW

SEQ ID NO: 74 - protein, 17 aa, unidentified
  REGION 1..17: note = TDD29 W819-C
IDKIKIYTGNNKIPKTN

SEQ ID NO: 75 - protein, 37 aa, unidentified
  REGION 1..37: note = TDD30 G195-N
KKKNANKQKIELPPYDGKTTYGVLILDDGKQYSFNSG

SEQ ID NO: 76 - protein, 92 aa, unidentified
  REGION 1..92: note = TDD30 G195-C
KPAPIYRNYIPASHVEGKAAIYMRENKIQSGTVYHNNTDGTCPYCDKMLPTLLEKDSTLKVVPPQNATSSKKGWITNEKIYIGNDKIPKTAR

SEQ ID NO: 77 - protein, 106 aa, unidentified
  REGION 1..106: note = TDD30 S264-N
KKKNANKQKIELPPYDGKTTYGVLILDDGKQYSFNSGKPAPIYRNYIPASHVEGKAAIYMRENKIQSGTVYHNNTDGTCPYCDKMLPTLLEKDSTLKVVPPQNATS

SEQ ID NO: 78 - protein, 23 aa, unidentified
  REGION 1..23: note = TDD30 S264-C
SKKGWITNEKIYIGNDKIPKTAR

SEQ ID NO: 79 - protein, 111 aa, unidentified
  REGION 1..111: note = TDD30 W269-N
KKKNANKQKIELPPYDGKTTYGVLILDDGKQYSFNSGKPAPIYRNYIPASHVEGKAAIYMRENKIQSGTVYHNNTDGTCPYCDKMLPTLLEKDSTLKVVPPQNATSSKKGW

SEQ ID NO: 80 - protein, 18 aa, unidentified
  REGION 1..18: note = TDD30 W269-C
ITNEKIYIGNDKIPKTAR

SEQ ID NO: 81 - protein, 41 aa, unidentified
  REGION 1..41: note = TDD31 G41-N
DMEAGLGESGRAHIALPEYDGGTTHGVLVLDDGTQIPLSSG

SEQ ID NO: 82 - protein, 97 aa, unidentified
  REGION 1..97: note = TDD31 G41-C
KGDPPGTNYAASGHVEGKAALIMRENGITSATLYINNPNGTCGYCDSQLPTLLPEGATLTVVPPANAVAPKSRWIDYPKTYIGNSNDPKPWPRYKGN

SEQ ID NO: 83 - protein, 115 aa, unidentified
  REGION 1..115: note = TDD31 W115-N
DMEAGLGESGRAHIALPEYDGGTTHGVLVLDDGTQIPLSSGKGDPPGTNYAASGHVEGKAALIMRENGITSATLYINNPNGTCGYCDSQLPTLLPEGATLTVVPPANAVAPKSRW

SEQ ID NO: 84 - protein, 23 aa, unidentified
  REGION 1..23: note = TDD31 W115-C
IDYPKTYIGNSNDPKPWPRYKGN

SEQ ID NO: 85 - protein, 26 aa, synthetic construct
  REGION 1..26: note = Synthetic: L26 linker
LRGSQLVKSKSEAAARGGGGSGGGGS

SEQ ID NO: 86 - protein, 21 aa, synthetic construct
  REGION 1..21: note = Synthetic: L21 linker
LRGSQLVKSKSEAAARGGGGS

SEQ ID NO: 87 - protein, 18 aa, synthetic construct
  REGION 1..18: note = Synthetic: L18 linker
LRGSQLVKSKSEAAARGS

SEQ ID NO: 88 - protein, 13 aa, synthetic construct
  REGION 1..13: note = Synthetic: L13 linker
LRGSQLVKSKSGS

SEQ ID NO: 89 - protein, 11 aa, synthetic construct
  REGION 1..11: note = Synthetic: L11 linker
LRGSQLVKSGS

SEQ ID NO: 90 - protein, 9 aa, synthetic construct
  REGION 1..9: note = Synthetic: L9 linker
LRGSQLVGS

SEQ ID NO: 91 - protein, 16 aa, synthetic construct
  REGION 1..16: note = Synthetic: L7A linker
LRGSQLVKSKSEAAAR

SEQ ID NO: 92 - protein, 6 aa, synthetic construct
  REGION 1..6: note = Synthetic: L6 linker
LRGSGS

SEQ ID NO: 93 - protein, 4 aa, synthetic construct
  REGION 1..4: note = Synthetic: L4 linker
LRGS

SEQ ID NO: 94 - protein, 9 aa, synthetic construct
  REGION 1..9: note = Synthetic: L0 linker
LRGSQLVKS

SEQ ID NO: 95 - protein, 4 aa, synthetic construct
  REGION 1..4: note = Synthetic: SGGS linker
SGGS

SEQ ID NO: 96 - protein, 10 aa, synthetic construct
  REGION 1..10: note = Synthetic: N6A linker
SGAQGSTLDF

SEQ ID NO: 97 - protein, 42 aa, synthetic construct
  REGION 1..42: note = Synthetic: 3xFlag+NLS
MDYKDHDGDYKDHDIDYKDDDDKMAPKKKRKVGIHGVPAAMA

SEQ ID NO: 98 - protein, 47 aa, synthetic construct
  REGION 1..47: note = Synthetic: 3xFlag+NLS (variant)
MDYKDHDGDYKDHDIDYKDDDDKMAPKKKRKVGIHGVPAAMGQLVKS

SEQ ID NO: 99 - protein, 83 aa, synthetic construct
  REGION 1..83: note = Synthetic: UGI
TNLSDIIEKETGKQLVIQESILMLPEEVEEVIGNKPESDILVHTAYDESTDENVMLLTSDAPEYKPWALVIQDSNGENKIKML

SEQ ID NO: 100 - protein, 171 aa, synthetic construct
  REGION 1..171: note = Synthetic: CIITA site 2 Left ZFP #6
RPFQCRICMRNFSRSAHLSRHIRTHTGEKPFACDICGRKFATSGHLSRHTKIHTHPRAPIPKPFQCRICMRNFSDSSHRTRHIRTHTGEKPFACDICGRKFAAKWNLDAHTKIHTGSQKPFQCRICMRNFSRPYTLRLHIRTHTGEKPFACDICGRKFALRHHLTRHTKIH

SEQ ID NO: 101 - protein, 171 aa, synthetic construct; identical to SEQ ID NO: 100
  REGION 1..171: note = Synthetic: CIITA site 2 Left ZFP #6 (variant)

SEQ ID NO: 102 - protein, 168 aa, synthetic construct
  REGION 1..168: note = Synthetic: CIITA site 2 Right ZFP #1
ERPFQCRICMRNFSQSGHLARHIRTHTGEKPFACDICGRKFARKWTLQGHTKIHTGSQKPFQCRICMRNFSIRSTLRDHIRTHTGEKPFACDICGRKFAHRSSLRRHTKIHTGSQKPFQCRICMRNFSQSGNLARHIRTHTGEKPFACDICGRKFARNVDLIHHTKIH

SEQ ID NO: 103 - protein, 168 aa, synthetic construct
  REGION 1..168: note = Synthetic: CIITA site 2 Right ZFP #5
ERPFQCRICMRNFSIRSTLRDHIRTHTGEKPFACDICGRKFAHRSSLRRHTKIHTGSQKPFQCRICMRNFSQSGNLARHIRTHTGEKPFACDICGRKFARNVDLIHHTKIHTGSQKPFQCRICMRNFSRSDVLSEHIRTHTGEKPFACDICGRKFATSGHLSRHTKIH

SEQ ID NO: 104 - DNA, 45 bp, synthetic construct
  misc_feature 1..45: note = Synthetic: CIITA site 2 forward primer
  source 1..45: mol_type = other DNA
acacgacgctcttccgatctnnnnctggggcagctgatcacatgt

SEQ ID NO: 105 - DNA, 39 bp, synthetic construct
  misc_feature 1..39: note = Synthetic: CIITA site 2 reverse primer
  source 1..39: mol_type = other DNA
gacgtgtgctcttccgatctcttccatcccctccccaag

SEQ ID NO: 106 - DNA, 141 bp, synthetic construct
  misc_feature 1..141: note = Synthetic: CIITA site 2 NGS amplicon
  source 1..141: mol_type = other DNA
nnnnctggggcagctgatcacatgttttctctgcagccttcccagaggagcttccggcagacctgaagcactggaagccaggtgtgcagggcaggtgggctggggttgggaagggtggatgccttggggaggggatggaag

SEQ ID NO: 107 - DNA, 20 bp, synthetic construct
  misc_feature 1..20: note = Synthetic: forward primer
  source 1..20: mol_type = other DNA
acacgacgctcttccgatct

SEQ ID NO: 108 - DNA, 18 bp, synthetic construct
  misc_feature 1..18: note = Synthetic: reverse primer
  source 1..18: mol_type = other DNA
gacgtgtgctcttccgat

SEQ ID NO: 109 - protein, 191 aa, synthetic construct
  REGION 1..191: note = Synthetic: FokI-ELD_D450N
ELEEKKSELRHKLKYVPHEYIELIEIARNSTQDRILEMKVMEFFMKVYGYRGKHLGGSRKPNGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 110 - protein, 193 aa, synthetic construct
  REGION 1..193: note = Synthetic: FokI-KKR
ELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFRS

SEQ ID NO: 111 - protein, 168 aa, synthetic construct
  REGION 1..168: note = Synthetic: CIITA_site_2_Nickase ZFP
ERPFQCRICMRNFSERGTLARHIRTHTGEKPFACDICGRKFATSANLSRHTKIHTGSQKPFQCRICMRNFSRSDVLSAHIRTHTGEKPFACDICGRKFARNDHRINHTKIHTGSQKPFQCRICMRNFSQSAHRKNHIRTHTGEKPFACDICGRKFARPYTLRLHTKIH

SEQ ID NO: 112 - protein, 238 aa, synthetic construct
  REGION 1..238: note = Synthetic: 3xFLAG-NLS-FokI-ELD_D450N
MDYKDHDGDYKDHDIDYKDDDDKMAPKKKRKVGIHGVPAAMGQLVKSELEEKKSELRHKLKYVPHEYIELIEIARNSTQDRILEMKVMEFFMKVYGYRGKHLGGSRKPNGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINF

SEQ ID NO: 113 - protein, 412 aa, synthetic construct
  REGION 1..412: note = Synthetic: ZFP-FokI-KKR
MDYKDHDGDYKDHDIDYKDDDDKMAPKKKRKVGIHGVPAAMAERPFQCRICMRNFSERGTLARHIRTHTGEKPFACDICGRKFATSANLSRHTKIHTGSQKPFQCRICMRNFSRSDVLSAHIRTHTGEKPFACDICGRKFARNDHRINHTKIHTGSQKPFQCRICMRNFSQSAHRKNHIRTHTGEKPFACDICGRKFARPYTLRLHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFRS

SEQ ID NO: 114 - DNA, 60 bp, synthetic construct
  misc_feature 1..60: note = Synthetic
  source 1..60: mol_type = other DNA
tcccagaggagcttccggcagacctgaagcactggaagccaggtgtgcagggcaggtggg

SEQ ID NO: 115 - DNA, 60 bp, synthetic construct
  misc_feature 1..60: note = Synthetic
  source 1..60: mol_type = other DNA
cccacctgccctgcacacctggcttccagtgcttcaggtctgccggaagctcctctggga

SEQ ID NO: 116 - DNA, 79 bp, synthetic construct
  misc_feature 1..79: note = Synthetic
  source 1..79: mol_type = other DNA
gaggagcttccggcagacctgaagcactggaagccaggtgtgcagggcaggtgggctggggttgggaagggtggatgcc

SEQ ID NO: 117 - DNA, 79 bp, synthetic construct
  misc_feature 1..79: note = Synthetic
  source 1..79: mol_type = other DNA
ggcatccacccttcccaaccccagcccacctgccctgcacacctggcttccagtgcttcaggtctgccggaagctcctc

SEQ ID NO: 118 - protein, 12 aa, synthetic construct
  REGION 1..12: note = Synthetic
EELPADLKHWKP

SEQ ID NO: 119 - protein, 5 aa, synthetic construct
  REGION 1..5: note = Synthetic
GGGGS